SEQUENCE LISTING (33 sequences)

SEQ ID NO: 1 - DNA, 217 bp, Lachnoclostridium sp.
  misc_feature: Lachnospiraceae Lachnoclostridium
gtaaagggagcgtagacggtaaagcaagtctgaagtgaaagcccggggctcaaccccgggactgctttggaaactgtttaactagagtgctggagaggtaagcggaattcctagtgtagcggtgaaatgcgtagatattaggaggaacaccagtggcgaaggcggcttactggacagtaactgacgttgaggctcgaaagcgtggggagcaaacagg

SEQ ID NO: 2 - DNA, 217 bp, Fusobacterium sp.
  misc_feature: Fusobacteriaceae Fusobacterium
cgtaaagcgcgtctaggcggtttggtaagtctgatgtgaaaatgcggggctcaactccgtattgcgttggaaactgccaaactagagtactggagaggtgggcggaactacaagtgtagaggtgaaattcgtagatatttgtaggaatgccaatggggaagccagcccactggacagatactgacgctaaagcgcgaaagcgtgggtagcaaacagg

SEQ ID NO: 3 - DNA, 217 bp, Solobacterium sp.
  misc_feature: Erysipelotrichaceae Solobacterium
cgtaaagggtgcgtaggcggcctgttaagtaagtggttaaattgttgggctcaacccaatccagccacttaaactggcaggctagagtattggagaggcaagtggaattccatgtgtagcggtaaaatgcgtagatatatggaggaacaccagtggcgaaggcggcttgctagccaaagactgacgctcatgcacgaaagcgtggggagcaaatagg

SEQ ID NO: 4 - DNA, 217 bp, Aggregatibacter sp.
  misc_feature: Pasteurellaceae Aggregatibacter
gtaaagggcacgcaggcggacttttaagtgaggtgtgaaatccccgggcttaacctgggaattgcatttcagactgggggtctagagtactttagggaggggtagaattccacgtgtagcggtgaaatgcgtagagatgtggaggaataccgaaggcgaaggcagccccttgggaatgtactgacgctcatgtgcgaaagcgtggggagcaaacagg

SEQ ID NO: 5 - DNA, 217 bp, Acetanaerobacterium sp.
  misc_feature: Ruminococcaceae Acetanaerobacterium
gtaaagggagcgtaggcggtttggtaagttgagtgtgaaatctaccggcttaactggtaggctgcgctcaaaactaccaaacttgagtgaagtagaggcaggcggaattcccggtgtagcggtggaatgcgtagatatcgggaggaacaccagtggcgaaggcggcctgctgggcttttactgacgctgatgctcgaaagcatggggagcaaacagg

SEQ ID NO: 6 - DNA, 217 bp, Hydrogenoanaerobacterium sp.
  misc_feature: Ruminococcaceae Hydrogenoanaerobacterium
tgtaaagggagcgtaggcgggaagacaagttgaatgttaaatctatcggctcaaccggtagccgcgttcaaaactgtttttcttgagtgaagtagaggttggcggaattcctagtgtagcggtgaaatgcgtagatattaggaggaacaccagtggcgaaggcggccaactgggcttttactgacgctgaggctcgaaagcgtggggagcaaacagg

SEQ ID NO: 7 - DNA, 217 bp, Mailhella sp.
  misc_feature: Desulfovibrionaceae Mailhella
gtaaagcgcatgtaggccgtgtggcaagttaggggtgaaatcccagggctcaaccttggaactgcctctaaaactaccatgcttgagtgcgagagaggatagcggaattccaggtgtaggagtgaaatccgtagatatctggaagaacatcagtggcgaaggcggctatctggctcgtaactgacgctgagatgcgaaagcgtgggtagcaaacagg

SEQ ID NO: 8 - DNA, 217 bp, Coprococcus sp.
  misc_feature: Lachnospiraceae Coprococcus_2
gtaaagggtgcgtaggtggtgagacaagtctgaagtgaaaatccggggcttaaccccggaactgctttggaaactgcctgactagagtacaggagaggtaagtggaattcctagtgtagcggtgaaatgcgtagatattaggaggaacaccagtggcgaaggcgacttactggactgctactgacactgaggcacgaaagcgtggggagcaaacagg

SEQ ID NO: 9 - DNA, 217 bp, Barnesiella sp.
  misc_feature: Barnesiellaceae Barnesiella
ttaaagggtgcgtaggcggcacgccaagtcagcggtgaaatttccgggctcaacccggactgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacagg

SEQ ID NO: 10 - DNA, 217 bp, Prevotella sp.
  misc_feature: Prevotellaceae Prevotellaceae_UCG-001
ttaaagggagcgcaggcggccttttaagcgtgacgtgaaatgccggggctcaaccttggaattgcgtcgcgaactggcgggcttgagtacgctcgaggcaggcggaattcgtggtgtagcggtgaaatgcttagatatcacgaggaaccccgattgcgaaggcagcctgccggggtgttactgacgctcatgctcgaaggtgcgggtatcgaacagg

SEQ ID NO: 11 - DNA, 217 bp, Anaerotruncus sp.
  misc_feature: Ruminococcaceae Anaerotruncus
tgtaaagggagcgtaggcgggatggcaagttggatgtttaaactaacggctcaactgttaggtgcatccaaaactgctgttcttgagtgaagtagaggcaggcggaattcctagtgtagcggtgaaatgcgtagatattaggaggaacaccagtggcgaaggcggcctgctgggctttaactgacgctgaggctcgaaagcgtggggagcaaacagg

SEQ ID NO: 12 - DNA, 217 bp, Erysipelotrichaceae sp.
  misc_feature: Erysipelotrichaceae Erysipelotrichaceae_UCG-003
cgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatcacgaagaactccgattgcgaaggcagcctgctaagctgcaactgacattgaggctcgaaagtgtgggtatcaaacagg

SEQ ID NO: 13 - DNA, 217 bp, Faecalitalea sp.
  misc_feature: Erysipelotrichaceae Faecalitalea
cgtaaagggtgcgtaggtggtgcattaagtctgaagtaaaagccagcagctcaactgctgtaagctttggaaactggtgtactagagtgcaggagagggcgatggaattccatgtgtagcggtaaaatgcgtagatatatggaggaacaccagtggcgaaggcggtcgcctggcctgtaactgacactgaggcacgaaagcgtggggagcaaatagg

SEQ ID NO: 14 - DNA, 217 bp, Lachnospiraceae sp.
  misc_feature: Lachnospiraceae GCA-900066575
gtaaagggagcgtaggcggcgacgcaagtcagaagtgaaagcccggggctcaactccgggactgcttttgaaactgcgttgctagattgcgggagaggcaagtggaattcctagtgtagcggtgaaatgcgtagatattaggaggaacaccagtggcgaaggcggcttgctggaccgtgaatgacgctgaggctcgaaagcgtggggagcaaacagg

SEQ ID NO: 15 - DNA, 217 bp, Ruminococcaceae sp.
  misc_feature: Ruminococcaceae Ruminococcaceae_UCG-008
gtaaagggcgagtaggcgggtcggcaagttgggagtgaaatgtcggggcttaaccccggaactgcttccaaaactgttgatcttgagtgatggagaggcaggcggaattcccagtgtagcggtgaaatgcgtagatattgggaggaacaccagtggcgaaggcggcctgctggacattaactgacgctgaggagcgaaagcgtggggagcaaacagg

SEQ ID NO: 16 - DNA, 217 bp, Tyzzerella sp.
  misc_feature: Lachnospiraceae Tyzzerella
gtaaagggtgagtaggcggcatggtaagttagatgtgaaagcccggggcttaaccccgggattgcatttaaaactatcaagctcgagttcaggagaggtaagcggaattcctagtgtagcggtgaaatgcgtagatattaggaagaacaccggtggcgaaggcggcttactggactgatactgacgctgaggcacgaaagcgtggggagcaaacagg

SEQ ID NO: 17 - DNA, 217 bp, Butyricicoccus sp.
  misc_feature: Ruminococcaceae Butyricicoccus
gtaaagggcgcgcaggcgggccggtaagttggaagtgaaatctatgggcttaacccataaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacagg

SEQ ID NO: 18 - DNA, 217 bp, Sutterella sp.
  misc_feature: Burkholderiaceae Sutterella
gtaaagggtgcgcaggcggctgtgcaagacagatgtgaaatccccgggcttaacctgggaactgcatttgtgactgcacggctagagtttgtcagaggagggtggaattccgcgtgtagcagtgaaatgcgtagatatgcggaagaacaccaatggcgaaggcagccctctgggacatgactgacgctcatgcacgaaagcgtggggagcaaacagg

SEQ ID NO: 19 - DNA, 217 bp, Catabacter sp.
  misc_feature: Christensenellaceae Catabacter
gtaaagggtgcgtaggtggccatgtaagttaggtgtgaaagaccggggcttaaccccggggcggcacttaaaactgtgtggcttgagtacaggagagggaagtggaattcctagtgtagcggtgaaatgcgtagatattaggaggaacaccagtggcgaaggcgactttctggactgtaactgacactgaggcacgaaagcgtggggagcaaacagg

SEQ ID NO: 20 - DNA, 217 bp, Oscillibacter sp.
  misc_feature: Ruminococcaceae Oscillibacter
gtaaagggcgtgtagccgggtcggcaagtcagatgtgaaatccacgggcttaacccgtgaactgcatttgaaactgctgatcttgagtgtcggagaggtaatcggaattcctagtgtagcggtgaaatgcgtagatattaggaagaacaccggtggcgaaggcggattactggacgataactgacggtgaggcgcgaaagcgtggggagcaaacagg

SEQ ID NO: 21 - DNA, 217 bp, Anaeroglobus sp.
  misc_feature: Veillonellaceae Anaeroglobus
gtaaagggcgcgcaggcggctgtgtaagtctgtctagaaagtgcggggctaaaccccgtgagaggatggaaactggacagctgagagtgtcggagaggaaagcggaattcctagtgtagcggtgaaatgcgtagatattaggaggaacaccggtggcgaaagcggctttctggacgacaactgacgctgaggcgcgaaagccaggggagcaaacggg

SEQ ID NO: 22 - DNA, 217 bp, Anaerofilum sp.
  misc_feature: Ruminococcaceae Anaerofilum
tgtaaagggagcgcaggcggagctgtaagttgggcgtcaaatctacgggcttaacccgtatccgcgctcaaaactgtggctcttgagtagtgcagaggtaggtggaattcccggtgtagcggtggaatgcgtagatatcgggaggaacaccagtggcgaaggcggcctactgggcaccaactgacgctgaggctcgaaagtatgggtagcaaacagg

SEQ ID NO: 23 - DNA, 217 bp, Candidatus Soleaferrea sp.
  misc_feature: Ruminococcaceae Candidatus_Soleaferrea
tgtaaagggagcgtaggcgggtacgcaagttgaatgtgaaaactaacggctcaaccgatagttgcgttcaaaactgcggatcttgagtgaagtagaggcaggcggaattcctagtgtagcggtaaaatgcgtagatattaggaggaacaccagtggcgaaggcggcctgctgggctttaactgacgctgaggctcgaaagtgtggggagcaaacagg

SEQ ID NO: 24 - DNA, 217 bp, Oribacterium sp.
  misc_feature: Lachnospiraceae Oribacterium
gtaaagggagcgtagacggaatggcaagtctgaagtgaaatacccgggctcaacctgggaactgctttggaaactgttgttctagagtgttggagaggtaagtggaattcctggtgtagcggtgaaatgcgtagatatcaggaagaacaccggaggcgaaggcggcttactggacaataactgacgttgaggctcgaaagcgtggggatcaaacagg

SEQ ID NO: 25 - DNA, 217 bp, Allisonella sp.
  misc_feature: Veillonellaceae Allisonella
cgtaaagcgcgcgcaggcggccgtgcaagtccatcttaaaagcgtggggcttaaccccatgaggggatggaaactgcatggctggagtgtcggaggggaaagtggaattcctagtgtagcggtgaaatgcgtagagattaggaagaacaccggtggcgaaggcgactttctagacgacaactgacgctgaggcgcgaaagcgtggggagcaaacagg

SEQ ID NO: 26 - DNA, 217 bp, Brochothrix sp.
  misc_feature: Listeriaceae Brochothrix
gtaaagcgcgcgcaggcggtctcttaagtctgatgtgaaagcccccggctcaaccggggagggtcattggaaactgggagacttgaggacagaagaggagagtggaattccaagtgtagcggtgaaatgcgtagatatttggaggaacaccagtggcgaaggcggctctctggtctgttactgacgctgaggcgcgaaagcgtggggagcaaacagg

SEQ ID NO: 27 - DNA, 217 bp, Wolbachia sp.
  misc_feature: Anaplasmataceae Wolbachia
gtaaagggcgcgtaggctgattaataagttaaaagtgaaatcccgaggcttaaccttggaattgcttttaaaactattaatctagagattgaaagaggatagaggaattcctgatgtagaggtaaaattcgtaaatattaggaggaacaccagtggcgaaggcgtctatctggttcaaatctgacgctgaggcgcgaaggcgtggggagcaaacagg

SEQ ID NO: 28 - DNA, 217 bp, Buchnera sp.
  misc_feature: Enterobacteriaceae Buchnera
gtaaagagctcgtaggcggtatattaagtcagatgtgaaatcccttggcttaacctaggaactgcatttgaaactgataaactagagtatcgtagagggaggtagaattctaggtgtagcggtgaaatgcgtagatatctggaggaatacctgtggcgaaagcgacctcctaaacgaatactgacgctgaggtgcgaaagcgtggggagcaaacagg

SEQ ID NO: 29 - DNA, 217 bp, Lachnospiraceae sp.
  misc_feature: Lachnospiraceae Lachnospiraceae_UCG-010
taaagggtgagtaggcggcatggcaagtaagatgtgaaagcccgaggcttaacctcgggattgcattttaaactgctaagctagagtacaggagaggaaagcggaattcctagtgtagcggtgaaatgcgtagatattaggaagaacaccagtggcgaaggcggctttctggactggaaactgacgctgaggcacgaaagcgtggggagcgaacagg

SEQ ID NO: 30 - DNA, 217 bp, Alcaligenes sp.
  misc_feature: Burkholderiaceae Alcaligenes
gtaaagcgtgtgtaggcggttcggaaagaaagatgtgaaatcccagggctcaaccttggaactgcatttttaactgccgagctagagtatgtcagaggggggtagaattccacgtgtagcagtgaaatgcgtagatatgtggaggaataccgatggcgaaggcagccccctgggataatactgacgctcagacacgaaagcgtggggagcaaacagg

SEQ ID NO: 31 - DNA, 217 bp, Erysipelatoclostridium sp.
  misc_feature: Erysipelotrichaceae Erysipelatoclostridium
cgtaaagagggagcaggcggcggcagaggtctgtggtgaaagactgaagcttaacttcagtaagccatagaaaccgggctgctagagtgcaggagaggatcgtggaattccatgtgtagcggtgaaatgcgtagatatatggaggaacaccagtggcgaaggcgacggtctggcctgtaactgacgctcattcccgaaagcgtggggagcaaacagg

SEQ ID NO: 32 - DNA, 217 bp, Coprococcus sp.
  misc_feature: Lachnospiraceae Coprococcus_3
gtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacagg

SEQ ID NO: 33 - DNA, 217 bp, Cardiobacterium sp.
  misc_feature: Cardiobacteriaceae Cardiobacterium
gtaaagcgcacgcaggcggttgcccaagtcagatgtgaaagccccgggcttaacctgggaactgcatttgaaactgggcgactagagtatgaaagaggaaagcggaatttccagtgtagcagtgaaatgcgtagatattggaaggaacaccgatggcgaaggcagctttctgggtcgatactgacgctcatgtgcgaaagcgtggggagcaaacagg

(19) United States
(12) Patent Application Publication
(10) Pub. No.: US 2022/0073996 A1
(43) Pub. Date: Mar. 10, 2022

(54) MODEL FOR PREDICTING TREATMENT RESPONSIVENESS BASED ON INTESTINAL MICROBIAL INFORMATION

(71) Applicant: SHENZHEN XBIOME BIOTECH CO., LTD., Guangdong (CN)

(72) Inventors: Han HU, Guangdong (CN); Yan TAN, Guangdong (CN)

(21) Appl. No.: 17/424,554

(22) PCT Filed: Jan. 14, 2020
(86) PCT No.: PCT/CN2020/072001

§ 371 Date: Jul. 21, 2021

(30) Foreign Application Priority Data
Jan. 22, 2019 (CN) 201910066127.6

(51) Int. Cl. C12Q 1/6886; C12Q 1/689; G16B 40/00

(52) U.S. Cl. CPC C12Q 1/6886; G16B 40/00; C12Q 1/689

(57) ABSTRACT

The present disclosure provides a method for predicting a responsiveness of a subject to treatment with an immune checkpoint inhibitor therapy such as a PD-1 signaling pathway inhibitor from a sample comprising the gut microbiota of the subject through the presence and abundance information of microorganisms of one or more genera. Also disclosed are sequences and compositions for detecting intestinal microorganisms, and related uses thereof.

### TECHNICAL FIELD

The present invention generally relates to the field of disease treatment. Specifically, the present invention relates to a method for predicting a responsiveness of a subject to treatment with an immune checkpoint inhibitor such as a PD-1/PD-L1 inhibitor by using intestinal microbial information. The present invention also relates to sequences and compositions for detecting intestinal microorganisms to implement the above methods, and related uses thereof.

### BACKGROUND ART

Surgery, chemotherapy and radiotherapy are the “troika” of traditional cancer treatment. However, these traditional methods generally have the characteristics of low cure rate, easy relapse, and large side effects. In recent years, immune checkpoint inhibitors (ICIs), represented by PD-1/PD-L1 inhibitors, have gradually become a rising star in cancer treatment. These drugs block the binding of the receptors and ligands of immune checkpoint molecules such as PD-1/PD-L1, CTLA-4, so as to effectively prevent the inhibitory effect of co-inhibitors on T cells and promote the further activation, proliferation and differentiation of T cells and ultimately achieve the elimination of tumor cells.

PD-1 (programmed death-1, programmed death receptor-1), which is a type of immune checkpoint molecule expressed by T cells, belongs to the CD28 superfamily. PD-1, as an important immunosuppressive molecule, functions as a “closed switch” to inhibit T cells from attacking other cells in the body. When the PD-1 on the surface of T cells binds to the PD-1 ligand PD-L1 (programmed death ligand-1) expressed on normal cells in the body, the cell killing effect of T cells is inhibited. Tumor cells use this mechanism to escape from the immune attack of T cells. They express a large amount of PD-L1 to bind to PD-1 on the surface of T cells and inhibit the cell killing effect of T cells. Inhibitors against PD-1 or PD-L1 immune checkpoint, such as monoclonal antibody drugs, can block the binding of PD-1 to PD-L1 and inhibit its downstream signal transduction, thereby enhancing the immune killing effect of T cells on tumor cells. Immunomodulation targeting PD-1 is of great significance in anti-tumor, anti-infection, anti-autoimmune diseases and organ transplant survival. According to current clinical research and preclinical research, PD-1 antibody drugs have shown significant effects in treatment of a variety of cancers, including a variety of digestive tract cancers, melanoma, non-small cell lung cancer, kidney cancer, etc. Some patients who receive PD-1 antibody therapy can obtain long-term and lasting curative effects.

However, immune checkpoint inhibitors represented by PD-1/PD-L1 inhibitors also have many problems in cancer treatment, among which the low responsiveness rate is the most prominent. Studies have shown that the responsiveness rate of patients treated with a drug targeting PD-1/PD-L1 is usually less than 40%, while the responsiveness rate of patients treated with ipilimumab, a CTLA-4 monoclonal antibody drug, is only about 15%, and some of the patients only responded locally. In addition, this type of treatment also has the following problems of: slow onset, with a median onset time of 12 weeks, which may delay the treatment time of patients; poor treatment effect for some patients; causing side effects in patients, for example, immune-related adverse events (irAEs) such as colitis, diarrhea, dermatitis, hepatitis and endocrine diseases, which may lead to early termination of the treatment; and expensive cost, which makes it difficult for ordinary patients to bear.

How to accurately screen the applicable patient population for immune checkpoint inhibitors such as PD-1/PD-L1 inhibitors, and how to enhance the effect of such inhibitors and expand the applicable population of the drugs, have become an urgent problem in clinical research. Although there are some indicators in the prior art for predicting the efficacy of PD-1/PD-L1 inhibitor drugs, such as PD-L1 expression level, MSI/dMMR, tumor mutational burden (TMB), etc., the performance of these indicators varies in various tumor types. TMB is currently a more commonly used indicator, but due to the different mutation rates of different types of cancers, the accuracy of predicting the responsiveness to receiving PD-1/PD-L1 inhibitor therapy in patients with different types of cancers by using TMB is also inconsistent. At present, the accuracy of its report is about 70%.

Therefore, there is still a need in the art for a new method for predicting patient's responsiveness to treatment with an immune checkpoint inhibitor such as a PD-1/PD-L1 inhibitor with high accuracy.

### DISCLOSURE OF INVENTION

For the purpose of explaining this specification, the following definitions will be applied, and when appropriate, singular terms also include their plural meanings, and vice versa. Unless otherwise stated, “or” means “and/or”. Unless otherwise stated or in the case where the use of “one or more” is clearly inappropriate, “one” herein means “one or more”. “comprising” and “including” are used interchangeably and is not intended to be limited. In addition, in the case where the term “comprising” is used in the description of one or more embodiments, a person skilled in the art will understand that said one or more embodiments may be described by using alternative terms “substantially consisting of” and/or “consisting of”.

The techniques used to manipulate nucleic acids, such as subcloning, labeling probes, sequencing, hybridization, etc., are well described in scientific and patent literatures, see, for example, MOLECULAR CLONING: A LABORATORY MANUAL (2ND ED.), edited by Sambrook, Vols. 1-3, Cold Spring Harbor Laboratory, (1989); CURRENT PROTOCOLS IN MOLECULAR BIOLOGY, edited by Ausubel, John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, edited by Tijssen, Elsevier, N.Y. (1993), each of which are incorporated herein by reference.

The nomenclature of microorganisms involved in the present invention is derived from the SILVA database, Version 132.

The present invention relates at least in part to predicting the subject's responsiveness to an immune checkpoint inhibitor therapy based on information about the subject's gut microbiota. The present inventors unexpectedly discovered that it is possible to predict subject's responsiveness to immune checkpoint inhibitor (such as PD-1/PD-L1) therapy with high accuracy by using the presence and abundance information of specific types of microorganisms in the gut microbiota of the subject, thus completing the present invention.

Method

Accordingly, in one aspect, the present invention relates to a method for identifying a responsiveness of a subject to immune checkpoint inhibitor therapy, comprising:

a) providing a sample comprising the gut microbiota of the subject;

b) detecting in the sample the presence and abundance information of microorganisms of one or more genera selected from the group consisting of genera listed in Table 1:

TABLE 1
Lachnospiraceae Lachnoclostridium
Fusobacteriaceae Fusobacterium
Erysipelotrichaceae Solobacterium
Pasteurellaceae Aggregatibacter
Ruminococcaceae Acetanaerobacterium
Ruminococcaceae Hydrogenoanaerobacterium
Desulfovibrionaceae Mailhella
Lachnospiraceae Coprococcus_2
Barnesiellaceae Barnesiella
Prevotellaceae Prevotellaceae_UCG-001
Ruminococcaceae Anaerotruncus
Erysipelotrichaceae Erysipelotrichaceae_UCG-003
Erysipelotrichaceae Faecalitalea
Lachnospiraceae GCA-900066575
Ruminococcaceae Ruminococcaceae_UCG-008
Lachnospiraceae Tyzzerella
Ruminococcaceae Butyricicoccus
Burkholderiaceae Sutterella
Christensenellaceae Catabacter
Ruminococcaceae Oscillibacter
Veillonellaceae Anaeroglobus
Ruminococcaceae Anaerofilum
Ruminococcaceae Candidatus_Soleaferrea
Lachnospiraceae Oribacterium
Veillonellaceae Allisonella
Listeriaceae Brochothrix
Anaplasmataceae Wolbachia
Enterobacteriaceae Buchnera
Lachnospiraceae Lachnospiraceae_UCG-010
Burkholderiaceae Alcaligenes
Erysipelotrichaceae Erysipelatoclostridium
Lachnospiraceae Coprococcus_3
Cardiobacteriaceae Cardiobacterium

c) identifying the subject's responsiveness to immune checkpoint inhibitor therapy based on the presence and abundance information of the microorganisms of the one or more genera.

In some embodiments, the immune checkpoint inhibitor is a CTLA-4 signaling pathway inhibitor. In some other embodiments, the immune checkpoint inhibitor is a PD-1 signaling pathway inhibitor.

In some embodiments, the inhibitor is selected from the group consisting of an antibody, an antibody fragment, a corresponding ligand or antibody, a fusion protein and a small molecule inhibitor. z

In some embodiments, the immune checkpoint inhibitor is a PD-1 signaling pathway inhibitor, and the PD-1 signaling pathway inhibitor is selected from the group consisting of a PD-1 inhibitor and a PD-L1 inhibitor.

In some embodiments, the PD-1 inhibitor may be selected from the group consisting of: ANA011, BGB-A317, KD033, pembrolizumab, MCLA-134, mDX400, MEDI0680, muDX400, nivolumab, PDR001, PF-06801591, Pembrolizumab, REGN-2810, SHR 1210, STI-A1110, TSR-042, ANB011, 244C8, 388D4 and XCE853, but not limited thereto.

In some embodiments, the PD-L1 inhibitor may be selected from the group consisting of: Aviruzumab, BMS-936559, CA-170, Devaluzumab, MCLA-145, SP142, STI-A1011, STI-A1012, STI-A1010, STI-A1014, A110, KY1003 and Atezolizumab, but not limited thereto.

In any embodiment, the subject is a mammal. Preferably, the mammal is a rat, a mouse, a cat, a dog, a horse or a primate. Most preferably, the mammal is a human.

In some embodiments of the above method, the subject has cancer. In some embodiments, the cancer is a digestive tract cancer. In other embodiments, the cancer may be selected from the group consisting of an esophageal cancer, a gastric cancer, an ampullary cancer, a colorectal cancer, a sarcoidosis, a pancreatic cancer, a nasopharyngeal cancer, a neuroendocrine tumor, a melanoma, a non-small cell lung cancer, a liver cancer and a kidney cancer.

In some embodiments, the cancer is a primary cancer. In other embodiments, the cancer is a metastatic cancer.

In some embodiments, the subject is receiving or preparing to receive the immune checkpoint inhibitor therapy.

In some embodiments, the sample may be a tissue in the body. Alternatively, the sample can be collected or isolated in vitro (e.g., a tissue extract). In some embodiments, the sample may be a cell-containing sample from a subject.

In some embodiments, the sample is an intestinal tissue sample of the subject. In other embodiments, the sample is a stool sample.

In some embodiments of the above method, the presence and abundance information of microorganisms of one or more genera, for example, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all 33 genera, selected from the group consisting of genera listed in Table 1 in the sample can be detected, and the responsiveness of the subject to immune checkpoint inhibitor therapy is identified through the above-mentioned presence and abundance information. For example, the presence and abundance information of microorganisms of 2-30 genera, 3-25 genera, 5-20 genera, or 10-18 genera selected from the group consisting of genera listed in Table 1 in the sample can be detected, and the subject's responsiveness to immune checkpoint inhibitor therapy can be identified by the above-mentioned presence and abundance information.

In a preferred embodiment, detecting the presence and abundance information of the microorganisms of the one or more genera includes detecting the presence and abundance information of microorganisms of at least one, for example, at least 2, at least 3, at least 4, at least 5, at least 6, at least 7, at least 8, at least 9, at least 10, at least 11, at least 12, at least 13, at least 14 genera, for example all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

In some embodiments, detecting the presence and abundance information of the microorganisms of the one or more genera includes detecting the presence and abundance information of microorganisms of all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea and Ruminococcaceae Ruminococcaceae_UCG-008.

In some embodiments, detecting the presence and abundance information of the microorganisms of the one or more genera includes detecting the presence and abundance information of microorganisms of all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

In some embodiments of the above method, the presence and abundance information of the microorganisms are detected by targeted sequencing analysis, metagenomic sequencing analysis or qPCR analysis. In some embodiments, the targeted sequencing analysis is 16s rDNA sequencing analysis.

In some embodiments, the presence and abundance information of the microorganisms of the one or more genera are detected by detecting the presence and abundance information of a nucleotide sequence having at least 70%, for example, at least 75%, at least 80%, at least 85%, at least 90%, or at least 95% of sequence identity to a nucleotide sequence shown in Table 2 or a fragment thereof:

TABLE 2

| | |
|---|---|
| Lachnospiraceae Lachnoclostridium | SEQ ID NO: 1 |
| Fusobacteriaceae Fusobacterium | SEQ ID NO: 2 |
| Erysipelotrichaceae Solobacterium | SEQ ID NO: 3 |
| Pasteurellaceae Aggregatibacter | SEQ ID NO: 4 |
| Ruminococcaceae Acetanaerobacterium | SEQ ID NO: 5 |
| Ruminococcaceae Hydrogenoanaerobacterium | SEQ ID NO: 6 |
| Desulfovibrionaceae Mailhella | SEQ ID NO: 7 |
| Lachnospiraceae Coprococcus_2 | SEQ ID NO: 8 |
| Barnesiellaceae Barnesiella | SEQ ID NO: 9 |
| Prevotellaceae Prevotellaceae_UCG-001 | SEQ ID NO: 10 |
| Ruminococcaceae Anaerotruncus | SEQ ID NO: 11 |
| Erysipelotrichaceae Erysipelotrichaceae_UCG-003 | SEQ ID NO: 12 |
| Erysipelotrichaceae Faecalitalea | SEQ ID NO: 13 |
| Lachnospiraceae GCA-900066575 | SEQ ID NO: 14 |
| Ruminococcaceae Ruminococcaceae_UCG-008 | SEQ ID NO: 15 |
| Lachnospiraceae Tyzzerella | SEQ ID NO: 16 |
| Ruminococcaceae Butyricicoccus | SEQ ID NO: 17 |
| Burkholderiaceae Sutterella | SEQ ID NO: 18 |
| Christensenellaceae Catabacter | SEQ ID NO: 19 |
| Ruminococcaceae Oscillibacter | SEQ ID NO: 20 |
| Veillonellaceae Anaeroglobus | SEQ ID NO: 21 |
| Ruminococcaceae Anaerofilum | SEQ ID NO: 22 |
| Ruminococcaceae Candidatus_Soleaferrea | SEQ ID NO: 23 |
| Lachnospiraceae Oribacterium | SEQ ID NO: 24 |
| Veillonellaceae Allisonella | SEQ ID NO: 25 |
| Listeriaceae Brochothrix | SEQ ID NO: 26 |
| Anaplasmataceae Wolbachia | SEQ ID NO: 27 |
| Enterobacteriaceae Buchnera | SEQ ID NO: 28 |
| Lachnospiraceae Lachnospiraceae_UCG-010 | SEQ ID NO: 29 |
| Burkholderiaceae Alcaligenes | SEQ ID NO: 30 |
| Erysipelotrichaceae Erysipelatoclostridium | SEQ ID NO: 31 |
| Lachnospiraceae Coprococcus_3 | SEQ ID NO: 32 |
| Cardiobacteriaceae Cardiobacterium | SEQ ID NO: 33 |

In some embodiments of the above method, in step c), the subject's responsiveness to immune checkpoint inhibitor therapy is identified by a machine learning method.

In some embodiments, the machine learning method is a random forest model or a logistic regression model. The random forest model or logistic regression model uses the presence and abundance information of microorganisms of one or more genera as a feature.

In some embodiments, the random forest model or logistic regression model further includes using the presence and abundance information of other types of microorganisms as a featured in.

In some embodiments, the random forest model or logistic regression model further includes using the subject's allergy history as a feature.

A person skilled in the art will understand that in addition to the history of allergy, other information of the subject can also be used as a feature to determine the subject's responsiveness to immune checkpoint inhibitor therapy. Exemplary subject information includes, for example:

Height;

Body weight;

Gender;

History of bowel disease;

Whether the subject ever had a fever or severe infection in the past four weeks;

Whether the subject received gastrointestinal surgery such as stomach surgery, small intestine surgery, large intestine surgery, appendectomy, gastric bypass, gastric band, etc. in the past six months;

Whether the subject took Chinese medicine in the past week;

Whether the subject ate foods such as probiotics or prebiotics in the past week;

Whether the subject had diarrhea in the past week;

Whether the subject ate spicy food in the past week;

Whether the subject has a history of smoking;

Whether the subject drinks alcohol regularly.

In some embodiments of the above method, the subject is identified as responsive or non-responsive to the immune checkpoint inhibitor therapy.

As used herein, the terms “identifying” and “predicting” do not mean that the result occurs with 100% certainty. On the contrary, it is intended to mean that the result is more likely to occur than not occur. The behavior used to “identify” or “predict” may include determining the likelihood of the result that is more likely to occur than not occur.

Preferably, the method of the present invention has an accuracy of at least 70%, for example, 71%, 72%, 73%, 74%, 75%, 76%, 77%, 78% or 79%, preferably 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the present invention has a specificity of at least 70%, for example, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

Use

In another aspect, the present invention relates to a use of a detection reagent in identification of a responsiveness of a subject to immune checkpoint inhibitor therapy, the detection reagent being used for detecting the presence and abundance information of microorganisms of one or more genera selected from the group consisting of genera listed in Table 1 in a sample comprising the gut microbiota of the subject, wherein the subject's responsiveness to immune checkpoint inhibitor therapy is identified through the presence and abundance information of the microorganisms of the one or more genera.

In yet another aspect, the present invention relates to a use of a detection reagent in preparation of a kit for identifying a responsiveness of a subject to immune checkpoint inhibitor therapy, the detection reagent being used for detecting the presence and abundance information of microorganisms of one or more genera selected from the group consisting of genera listed in Table 1 in the sample comprising the gut microbiota of the subject, wherein the subject's responsiveness to immune checkpoint inhibitor therapy is identified through the presence and abundance information of the microorganisms of the one or more genera.

In some embodiments of the above uses, the immune checkpoint inhibitor is a CTLA-4 signaling pathway inhibitor. In some other embodiments, the immune checkpoint inhibitor is a PD-1 signaling pathway inhibitor.

In some embodiments, the inhibitor is selected from the group consisting of an antibody, an antibody fragment, a corresponding ligand or antibody, a fusion protein and a small molecule inhibitor.

In some embodiments, the PD-1 signaling pathway inhibitor is selected from the group consisting of a PD-1 inhibitor and a PD-L1 inhibitor.

In some embodiments, the PD-1 inhibitor may be selected from the group consisting of: ANA011, BGB-A317, KD033, pembrolizumab, MCLA-134, mDX400, MEDI0680, muDX400, nivolumab, PDR001, PF-06801591, Pembrolizumab, REGN-2810, SHR 1210, STI-A1110, TSR-042, ANB011, 244C8, 388D4 and XCE853, but not limited thereto.

In some embodiments, the PD-L1 inhibitor may be selected from the group consisting of: Aviruzumab, BMS-936559, CA-170, Devaluzumab, MCLA-145, SP142, STI-A1011, STI-A1012, STI-A1010, STI-A1014, A110, KY1003 and Atezolizumab, but not limited thereto.

In any embodiment, the subject is a mammal. Preferably, the mammal is a rat, a mouse, a cat, a dog, a horse or a primate. Most preferably, the mammal is a human.

In some embodiments of the above uses, the subject has cancer. In some embodiments, the cancer is a digestive tract cancer. In other embodiments, the cancer may be selected from the group consisting of an esophageal cancer, a gastric cancer, an ampullary cancer, a colorectal cancer, a sarcoidosis, a pancreatic cancer, a nasopharyngeal cancer, a neuroendocrine tumor, a melanoma, a non-small cell lung cancer, a liver cancer and a kidney cancer.

In some embodiments, the cancer is a primary cancer. In other embodiments, the cancer is a metastatic cancer.

In some embodiments, the subject is receiving or preparing to receive the immune checkpoint inhibitor therapy.

In some embodiments, the sample may be a tissue in the body. Alternatively, the sample can be collected or isolated in vitro (e.g., a tissue extract). In some embodiments, the sample may be a cell-containing sample from a subject.

In some embodiments, the sample is an intestinal tissue sample of the subject. In other embodiments, the sample is a stool sample.

In some embodiments of the above uses, the presence and abundance information of microorganisms of one or more genera, for example, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all 33 genera, selected from the group consisting of genera listed in Table 1 in the sample can be detected, and the responsiveness of the subject to immune checkpoint inhibitor therapy is identified through the above-mentioned presence and abundance information. For example, the presence and abundance information of microorganisms of 2-30 genera, 3-25 genera, 5-20 genera, or 10-18 genera selected from the group consisting of genera listed in Table 1 in the sample can be detected, and the subject's responsiveness to immune checkpoint inhibitor therapy can be identified by the above-mentioned presence and abundance information.

In a preferred embodiment of the above uses, detecting the presence and abundance information of the microorganisms of the one or more genera includes detecting the presence and abundance information of microorganisms of at least one, for example, at least 2, at least 3, at least 4, at least 5, at least 6, at least 7, at least 8, at least 9, at least 10, at least 11, at least 12, at least 13, at least 14 genera, for example all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

In some embodiments, detecting the presence and abundance information of the microorganisms of the one or more genera includes detecting the presence and abundance information of microorganisms of all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea and Ruminococcaceae Ruminococcaceae_UCG-008.

In some embodiments, detecting the presence and abundance information of the microorganisms of the one or more genera includes detecting the presence and abundance information of microorganisms of all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

A person skilled in the art will understand that the detection reagent may be any detection reagent capable of detecting the presence and abundance information of the microorganism. In some embodiments, the detection reagent comprises or consists of nucleic acid molecules. In other embodiments, the detection reagents each comprise or consist of DNA, RNA, PNA, LNA, GNA, TNA, or PMO. Preferably, the detection reagents each comprise or consist of DNA. In some embodiments, the length of the detection reagent is 5 to 100 nucleotides. However, in another embodiment, the length of the detection reagent is 15 to 35 nucleotides.

In some embodiments, the presence and abundance information of the microorganisms of the one or more genera is detected by detecting the presence and abundance information of the genomic DNA of the microorganisms of the one or more genera by using the detection reagent.

Preferred methods for nucleic acid detection and/or measurement include northern blotting, polymerase chain reaction (PCR), reverse transcriptase PCR (RT-PCR), quantitative real-time PCR (qRT-PCR), nanoarrays, microarrays, macroarrays, autoradiography and in situ hybridization.

In some embodiments of the above uses, the detection reagents are specific primers for the genomic DNA of the microorganisms of the one or more genera. In some embodiments, the primers are specific primers or qPCR primers for 16s rDNA of microorganisms of the one or more genera.

As known to a person skilled in the art, the term “primer” is used herein, and the term “primer” refers to an oligomeric compound, mainly oligonucleotide, but also refers to a modified oligonucleotide, which is capable of starting DNA synthesis through template-dependent DNA polymerase. That is, the 3′-end of the primer provides a free 3′-OH group, and a 3′- to 5′-phosphodiester bond is connected to the 3′-OH group through the template-dependent DNA polymerase, wherein pyrophosphate is released by using deoxy and nucleoside triphosphate. As used herein, the term “primer” refers to a continuous sequence, which in some embodiments contains about 6 or more nucleotides, in some embodiments about 10-20 nucleotides (e.g., 15-mer), and in some embodiments about 20-30 nucleotides (e.g., 22-mer). The primers used to implement the methods of the disclosed subject matter of the present invention encompass oligonucleotides with sufficient length and appropriate sequence to provide the initiation of polymerization on the nucleic acid molecule.

In some embodiments in which the primers are used as detection reagents, the presence and abundance information of microorganisms of the one or more genera is obtained by a PCR reaction using the primers and using the genomic DNA of the subject's gut microbiota as a template.

The method of nucleic acid amplification is polymerase chain reaction (PCR) well known to a person skilled in the art. Other amplification reactions include ligase chain reaction, polymerase ligase chain reaction, gap-LCR, repair chain reaction, 3SR, NASBA, strand displacement amplification (SDA), transcription-mediated amplification (TMA) and Qβ-amplification.

Automated systems for PCR-based analysis typically utilize real-time detection of product amplification during the PCR process in the same reaction vessel. The key to this method is the use of modified oligonucleotide that carries a reporter group or label.

A “label”, usually called a “reporter group”, is usually a group that distinguishes nucleic acids, especially oligonucleotide or modified oligonucleotide, bound to it, and any nucleic acid bound to it from the rest from the sample (nucleic acid to which the label is attached can also be referred to as labeled nucleic acid binding compound, labeled probe, or just probe). In some embodiments, the label is a fluorescent label, and may be a fluorescent dye, such as fluorescein dye, rhodamine dye, cyanine dye, and coumarin dye. Useful fluorescent dyes include FAM, HEX, JA270, CAL635, Coumarin343, Quasar705, Cyan500, CY5.5, LC-Red 640, LC-Red 705.

In some embodiments of the above uses, the presence and abundance information of the microorganisms of the one or more genera are detected by using the detection reagent to detect the presence and abundance information of a nucleotide sequence having at least 70%, for example, at least 75%, at least 80%, at least 85%, at least 90%, or at least 95% of sequence identity to a nucleotide sequence shown in Table 2 or a fragment thereof.

In some embodiments of the above uses, the identification of the subject's responsiveness to immune checkpoint inhibitor therapy through the presence and abundance information of the microorganisms of the one or more genera includes using a machine learning method.

In some embodiments, the machine learning method is a random forest model or a logistic regression model. The random forest model or logistic regression model uses the presence and abundance information of the microorganisms of the one or more genera as a feature.

In some embodiments, the random forest model or logistic regression model further includes using the presence and abundance information of other types of microorganisms as a feature.

In some embodiments, the random forest model or logistic regression model further includes using the subject's allergy history as a feature.

In some embodiments, the random forest model or logistic regression model further includes using other parameters of the subject as a feature. Exemplary parameters include, for example:

Height;

Body weight;

Gender;

History of bowel disease;

Whether the subject ever had a fever or severe infection in the past four weeks;

Whether the subject received gastrointestinal surgery such as stomach surgery, small intestine surgery, large intestine surgery, appendectomy, gastric bypass, gastric band, etc. in the past six months;

Whether the subject took Chinese medicine in the past week;

Whether the subject ate foods such as probiotics or prebiotics in the past week;

Whether the subject had diarrhea in the past week;

Whether the subject ate spicy food in the past week;

Whether the subject has a history of smoking;

Whether the subject drinks alcohol regularly.

In some embodiments of the above uses, the subject is identified as responsive or non-responsive to the immune checkpoint inhibitor therapy.

Kit

In another aspect, the present invention relates to a kit for identifying a responsiveness of a subject to immune checkpoint inhibitor therapy, the kit containing a detection reagent for detecting the presence and abundance information of microorganisms of one or more genera selected from the group consisting of genera listed in Table 1 in a sample comprising the gut microbiota of the subject.

In some embodiments of the above kit, the immune checkpoint inhibitor is a CTLA-4 signaling pathway inhibitor. In other embodiments, the immune checkpoint inhibitor is a PD-1 signaling pathway inhibitor.

In some embodiments, the inhibitor is selected from the group consisting of an antibody, an antibody fragment, a corresponding ligand or antibody, a fusion protein and a small molecule inhibitor.

In some embodiments, the PD-1 signaling pathway inhibitor is selected from the group consisting of a PD-1 inhibitor and a PD-L1 inhibitor.

In some embodiments, the PD-1 inhibitor may be selected from the group consisting of: ANA011, BGB-A317, KD033, pembrolizumab, MCLA-134, mDX400, MEDI0680, muDX400, nivolumab, PDR001, PF-06801591, Pembrolizumab, REGN-2810, SHR 1210, STI-A1110, TSR-042, ANB011, 244C8, 388D4 and XCE853, but not limited thereto.

In some embodiments, the PD-L1 inhibitor may be selected from the group consisting of: Aviruzumab, BMS-936559, CA-170, Devaluzumab, MCLA-145, SP142, STI-A1011, STI-A1012, STI-A1010, STI-A1014, A110, KY1003 and Atezolizumab, but not limited thereto.

In any embodiment, the subject is a mammal. Preferably, the mammal is a rat, a mouse, a cat, a dog, a horse or a primate. Most preferably, the mammal is a human.

In some embodiments of the above uses, the subject has cancer. In some embodiments, the cancer is a digestive tract cancer. In other embodiments, the cancer may be selected from the group consisting of an esophageal cancer, a gastric cancer, an ampullary cancer, a colorectal cancer, a sarcoidosis, a pancreatic cancer, a nasopharyngeal cancer, a neuroendocrine tumor, a melanoma, a non-small cell lung cancer, a liver cancer and a kidney cancer.

In some embodiments, the cancer is a primary cancer. In some other embodiments, the cancer is a metastatic cancer.

In some embodiments, the subject is receiving or preparing to receive the immune checkpoint inhibitor therapy.

In some embodiments, the sample may be a tissue in the body. Alternatively, the sample can be collected or isolated in vitro (e.g., a tissue extract). In some embodiments, the sample may be a cell-containing sample from a subject.

In some embodiments, the sample is an intestinal tissue sample of the subject. In other embodiments, the sample is a stool sample.

In some embodiments of the above kit, the presence and abundance information of microorganisms of one or more genera, for example, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or all 33 genera, selected from the group consisting of genera listed in Table 1 in the sample can be detected, and the responsiveness of the subject to immune checkpoint inhibitor therapy is identified through the above-mentioned presence and abundance information. For example, the presence and abundance information of microorganisms of 2-30 genera, 3-25 genera, 5-20 genera, or 10-18 genera selected from the group consisting of genera listed in Table 1 in the sample can be detected, and the subject's responsiveness to immune checkpoint inhibitor therapy can be identified by the above-mentioned presence and abundance information.

In a preferred embodiment of the above kit, detecting the presence and abundance information of the microorganisms of the one or more genera includes detecting the presence and abundance information of microorganisms of at least one, for example, at least 2, at least 3, at least 4, at least 5, at least 6, at least 7, at least 8, at least 9, at least 10, at least 11, at least 12, at least 13, at least 14 genera, for example all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

In some embodiments, detecting the presence and abundance information of the microorganisms of the one or more genera includes detecting the presence and abundance information of microorganisms of all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea and Ruminococcaceae Ruminococcaceae_UCG-008.

In some embodiments, detecting the presence and abundance information of the microorganisms of the one or more genera includes detecting the presence and abundance information of microorganisms of all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

A person skilled in the art will understand that the detection reagent may be any detection reagent capable of detecting the presence and abundance information of the microorganism. In some embodiments, the detection reagent comprises or consists of nucleic acid molecules. In other embodiments, the detection reagents each comprise or consist of DNA, RNA, PNA, LNA, GNA, TNA, or PMO Preferably, the detection reagents each comprise or consist of DNA. In some embodiments, the length of the detection reagent is 5 to 100 nucleotides. However, in another embodiment, the length of the detection reagent is 15 to 35 nucleotides.

In some embodiments, the presence and abundance information of the microorganisms of the one or more genera is detected by detecting the presence and abundance information of the genomic DNA of the microorganisms of the one or more genera by using the detection reagent.

In some embodiments of the above kit, the detection reagents are specific primers for the genomic DNA of the microorganisms of the one or more genera. In some embodiments, the primers are specific primers or qPCR primers for 16s rDNA of microorganisms of the one or more genera.

In some embodiments, the presence and abundance information of microorganisms of the one or more genera is obtained by a PCR reaction using the primers and using the genomic DNA of the subject's gut microbiota as a template.

In some embodiments of the above kit, the presence and abundance information of the microorganisms of the one or more genera are detected by detecting the presence and abundance information of a nucleotide sequence having at least 70%, for example, at least 75%, at least 80%, at least 85%, at least 90%, or at least 95% of sequence identity to a nucleotide sequence shown in Table 2 or a fragment thereof.

In any embodiment of the above kit, the kit further includes an instruction that describes the method for identifying the subject's responsiveness to immune checkpoint inhibitor therapy through the presence and abundance information of microorganisms of the one or more genera.

In some embodiments of the above kit, the method described in the instruction includes use of a machine learning method to identify the subject's responsiveness to immune checkpoint inhibitor therapy.

In some embodiments, the machine learning method is a random forest model or a logistic regression model. The random forest model or logistic regression model uses the presence and abundance information of microorganisms of one or more genera as a feature.

In some embodiments, the random forest model or logistic regression model further includes using the presence and abundance information of other types of microorganisms as a feature.

In some embodiments, the random forest model or logistic regression model further includes using the subject's allergy history as a feature.

In some embodiments, the random forest model or logistic regression model further includes using other parameters of the subject as a feature. Exemplary parameters includes, for example:

Height;

Body weight;

Gender;

History of bowel disease;

Whether the subject ever had a fever or severe infection in the past four weeks;

Whether the subject received gastrointestinal surgery such as stomach surgery, small intestine surgery, large intestine surgery, appendectomy, gastric bypass, gastric band, etc. in the past six months;

Whether the subject took Chinese medicine in the past week;

Whether the subject ate foods such as probiotics or prebiotics in the past week;

Whether the subject had diarrhea in the past week;

Whether the subject ate spicy food in the past week;

Whether the subject has a history of smoking;

Whether the subject drinks alcohol regularly.

In some embodiments of the above kit, the subject is identified as responsive or non-responsive to the immune checkpoint inhibitor therapy.

In some embodiments of the above kit, the kit further include a buffer, an enzyme, dNTPs and other components for performing PCR reaction.

A person skilled in the art will recognize that, in addition to the components specifically mentioned herein, the kit of the present invention may include other conventional substances in the art as needed.

### MODE FOR CARRYING OUT THE INVENTION

The invention is further illustrated by referring to the following examples. However, it should be noted that these examples are as illustrative as the above-mentioned embodiments and should not be construed as limiting the scope of the present invention in any way.

### Example 1. Data Collection and Model Generation

Sample collection, sequencing and data generation:

After the cancer patients signed the informed consent form, the stool samples of the cancer patients before receiving PD-1 immunotherapy were collected. After the patients received PD-1 immunotherapy under the guidance of the doctor, the corresponding tumor progress evaluation information (RECIST 1.1 standard) was collected. The method of receiving PD-1 immunotherapy is injection of a PD-1 antibody drug such as Keytruda. According to the RECIST 1.1 standard, the evaluation of patients can be divided into CR (complete response), PR (partial response), SD (stable disease) and PD (progressive disease, progressive development). The patient's response to PD-1 was marked as responsive (CR+PR) and non-responsive (PD); since the SD status is an intermediate state, for patient whose evaluation information is SD, it is necessary to combine multiple evaluation information to determine whether it is a stable SD state. If the SD state changes to an other state, it will be marked as the other state. If it is a stable SD state (all three consecutive evaluations are SD), the SD will also be marked as responsiveness.

The samples used included stool samples from 50 cancer patients. Among them, patients with esophageal cancer and gastric cancer accounted for the highest proportion, which together accounted for 60% of the total samples, colon cancer patients accounted for 14%, and other patients were approximately evenly dispersed in the other 9 kind of cancers.

The corresponding diagnosis information of the patients was shown in Table 3, and the statistics on the number of samples of various cancers were shown in Table 4. The samples were stored in a dedicated sampling tube and frozen at −80° C. before use.

TABLE 3
Corresponding diagnosis information table of the patients

| Sample number | Diagnosis |
|---|---|
| BD-QCS-0207 | esophageal cancer |
| BD-YM-0503 | ampullary cancer |
| BD-SQ-0308 | esophageal cancer |
| BD-HFS-0502 | gastric cancer |
| BD-HLT-0605 | neuroendocrine tumor |
| BD-LZH-0301 | small-bowel adenocarcinoma |
| BD-LBZ-0606 | intrahepatic cholangiocarcinoma |
| BD-LJZ-0323 | esophageal cancer |
| BD-LRH-0523 | gastric cancer |
| BD-LLY-0530 | gastric cancer |
| BD-LL-0403 | lung cancer |
| BD-WXJ-0412 | gastric cancer |
| BD-YMC-0213 | gastric cancer |
| BD-ZBL-0228 | gastric cancer |
| BD-ZXB-0326 | sarcoidosis |
| BD-ZZC-0428 | esophageal cancer |
| BD-ZCW-0529 | gastric cancer |
| BD-ZQA-0524 | esophageal cancer |
| BD-ZLY-0604 | gastric cancer |
| BD-PJL-0523 | gastric cancer |
| BD-XBQ-0305 | esophageal cancer |
| BD-LY-0604 | neuroendocrine tumor |
| BD-LSW-0314 | esophageal cancer |
| BD-LYX-0606 | colon cancer |
| BD-LQR-0426 | neuroendocrine tumor |
| BD-LDG-0606 | colon cancer |
| BD-DK-0307 | gastric cancer |
| BD-YZQ-0201 | gastric cancer |
| BD-KL-0522 | nasopharyngeal cancer |
| BD-DCY-0308 | colon cancer |
| BD-SYJ-0316 | colon cancer |
| BD-JSZ-0427 | gastric cancer |
| BD-WQL-0308 | esophageal cancer |
| BD-WJC-0522 | esophageal cancer |
| BD-WJC-0524 | esophageal cancer |
| BD-WJ-0322 | gastric cancer |
| BD-SYC-0411 | colon cancer |
| BD-QXY-0212 | gastric cancer |
| BD-ZML-0207 | colon cancer |
| BD-FGL-0209 | colon cancer |
| BD-DXZ-0601 | esophageal cancer |
| BD-LMR-0315 | neuroendocrine tumor |
| BD-ZWB-0326 | gastric cancer |
| BD-LJD-0426 | esophageal cancer |
| BD-SCL-0409 | abdominal |
| BD-GFC-0419 | esophagogastric junction carcinoma |
| BD-YJS-0606 | gastric cancer |
| BD-CJR-0607 | gastric cancer |
| BD-RXY-0307 | nasopharyngeal cancer |
| BD-LJS-0605 | gastric cancer |

TABLE 4
Types and number of cancers in patients

| | Number |
|---|---|
| Type of cancer | of samples |
| colon cancer | 7 |
| esophageal cancer | 12 |
| gastric cancer | 18 |
| esophagogastric junction carcinoma | 1 |
| liver cancer | 1 |
| nasopharyngeal cancer | 2 |
| neuroendocrine tumor | 4 |
| sarcoidosis | 1 |
| ampullary cancer | 1 |
| small-bowel adenocarcinoma | 1 |
| abdominal sarcoma | 1 |
| intrahepatic cholangiocarcinoma | 1 |

The bacterial genomic DNA in the sample was extracted and 16S rDNA sequencing was performed to obtain the composition of the bacteria and the abundance information of the bacteria in the sample. For 16S rDNA sequencing, primers for V4 or V3-V4 region of 16S rDNA were used for amplification, and the library was constructed after passing the quality inspection, and then the sequencing was perform. The sequencing data results were in fastq format. Each sample has a corresponding paired-end fastq file.

Data Preprocessing:

DADA2 (https://benjjneb.github.io/dada2/tutorial.html) was used to preprocess the 16S data. The basic process includes correcting sequencing errors in the 16S data and filtering low-quality short-read sequences. SILVA (v132 or v138) database and RDP algorithm (https://github.com/rdpstaff/classifier) were used to classify and quantify the preprocessed short-read sequences. The number of short-read sequences identified as the species by the classification was combined into the genus.

After above data processing, the result is the abundance (Cᵢⱼ, the number of the j^{th} bacteria in the i^{th} sample) of bacterial genera in respective samples. Then normalization was carried out to convert the abundance of bacterial genera in respective samples to relative abundance (Pᵢⱼ=Cᵢⱼ/ΣC_{i*}).

Prediction:

The samples were randomly divided into 3 groups (the three groups respectively included 16 samples, 16 samples, and 18 samples), and the ratio of R to NR of the corresponding subjects in each group of samples was approximated. One group was used as the test set, and the other two groups were used as the training set. The method of repeated sampling was adopted in the training set to make the numbers of NR and R consistent. The glmnet model was used to build a classifier.

For a sample i, the relative abundance of bacteria of the relevant genus was extracted from the above analysis results (the name was named using the SILVA database), and log conversion was performed:

Rij=log(1000*Pij+1)

wherein Pᵢⱼ is the relative abundance of bacteria j in the sample i.

For model 1, the weighted linear combination of bacteria in sample i was calculated:

yᵢ₁=intercept₁+Σⱼ₌₁ⁿ(Weightⱼ₁×Rᵢⱼ)

where j is the serial number of the bacteria, intercept₁ corresponds to the Intercept value in model 1, Weightⱼ₁ corresponds to the parameter value of model 1 of the genus of the bacteria with the serial number of j. Rᵢⱼ is the log conversion of the relative abundance of the bacteria with the serial number of j in the sample i.

The sigmoid function was used to project the above result to the interval (0, 1):

$${S}_{i 1} = \frac{1}{1 + {e}^{{y}_{i 1}}}$$

Similarly, the parameters of model 2 and model 3 were used to respectively calculate Sᵢ₂ and Sᵢ₃ in the same sample i.

S=(Sᵢ₁+Sᵢ₂+Sᵢ₃)/3

If S≥0.5, the patient corresponding to the sample was predicted to be responsive to immunotherapy, and if S<0.5, the patient corresponding to the sample was predicted to be non-responsive to immunotherapy.

Through screening, it was found that the presence and abundance information of the following bacterial genera in the sample can be used to accurately predict the patient's responsiveness to PD-1 immunotherapy.

TABLE 5
Bacteria used to predict patient's responsiveness
Lachnospiraceae Lachnoclostridium
Fusobacteriaceae Fusobacterium
Erysipelotrichaceae Solobacterium
Pasteurellaceae Aggregatibacter
Ruminococcaceae Acetanaerobacterium
Ruminococcaceae Hydrogenoanaerobacterium
Desulfovibrionaceae Mailhella
Lachnospiraceae Coprococcus_2
Barnesiellaceae Barnesiella
Prevotellaceae Prevotellaceae_UCG-001
Ruminococcaceae Anaerotruncus
Erysipelotrichaceae Erysipelotrichaceae_UCG-003
Erysipelotrichaceae Faecalitalea
Lachnospiraceae GCA-900066575
Ruminococcaceae Ruminococcaceae_UCG-008
Lachnospiraceae Tyzzerella
Ruminococcaceae Butyricicoccus
Burkholderiaceae Sutterella
Christensenellaceae Catabacter
Ruminococcaceae Oscillibacter
Veillonellaceae Anaeroglobus
Ruminococcaceae Anaerofilum
Ruminococcaceae Candidatus_Soleaferrea
Lachnospiraceae Oribacterium
Veillonellaceae Allisonella
Listeriaceae Brochothrix
Anaplasmataceae Wolbachia
Enterobacteriaceae Buchnera
Lachnospiraceae Lachnospiraceae_UCG-010
Burkholderiaceae Alcaligenes
Erysipelotrichaceae Erysipelatoclostridium
Lachnospiraceae Coprococcus_3
Cardiobacteriaceae Cardiobacterium

### Example 2. Prediction of Responsiveness Using the Presence and Abundance Information of the Bacteria

After DADA2 processing, 15 bacterial genera (selected from Table 5) as shown in Table 6 were used as features and their weight values were calculated.

TABLE 6
Summary of model features and parameters

| | | Model 1 | Model 2 | Model 3 |
|---|---|---|---|---|
| j | Feature | Weight | Weight | Weight |
| | Intercept | 0.036926644 | −0.003347488 | −0.003354876 |
| 1 | Lachnospiraceae | 0.314113103 | 0.223356499 | 0.103902521 |
| | Lachnoclostridium | | | |
| 2 | Fusobacteriaceae | 0.420712215 | 0.175687273 | 0.205407459 |
| | Fusobacterium | | | |
| 3 | Erysipelotrichaceae | −0.139211989 | −0.130704271 | −0.124890972 |
| | Solobacterium | | | |
| 4 | Pasteurellaceae | −0.370514801 | −0.075533452 | −0.181609972 |
| | Aggregatibacter | | | |
| 5 | Ruminococcaceae | −0.506365199 | −0.11502412 | −0.082069412 |
| | Acetanaerobacterium | | | |
| 6 | Ruminococcaceae | 0.255802661 | −0.125871575 | −0.060165451 |
| | Hydrogenoanaerobacterium | | | |
| 7 | Desulfovibrionaceae | −0.650499205 | −0.168939616 | −0.131568569 |
| | Mailhella | | | |
| 8 | Lachnospiraceae | −0.155061346 | −0.17549134 | −0.207819915 |
| | Coprococcus_2 | | | |
| 9 | Barnesiellaceae | −0.722041055 | −0.119440087 | −0.207316616 |
| | Barnesiella | | | |
| 10 | Prevotellaceae | 0 | −0.038505868 | −0.180359808 |
| | Prevotellaceae_UCG-001 | | | |
| 11 | Ruminococcaceae | 0 | 0.017024421 | −0.008546691 |
| | Anaerotruncus | | | |
| 12 | Erysipelotrichaceae | −0.437145184 | −0.059416751 | −0.120237538 |
| | Erysipelotrichaceae_UCG-003 | | | |
| 13 | Erysipelotrichaceae | 0 | −0.096912346 | −0.049348806 |
| | Faecalitalea | | | |
| 14 | Lachnospiraceae | 0.38077419 | 0.141513335 | 0 |
| | GCA-900066575 | | | |
| 15 | Ruminococcaceae | −0.190356893 | −0.202202515 | −0.117594401 |
| | Ruminococcaceae_UCG-008 | | | |

Note:
Each parameter in the model came from the training set data. The model was trained and constructed through the training of the training set data, and used to predict the test set data.

Using the features and weight in Table 6, the model prediction results were calculated by the formulae shown in Example 1, and shown in Table 7 below.

TABLE 7
Model prediction results

| | | Model 1 | Model 2 | Model 3 | Predicted |
|---|---|---|---|---|---|
| | | predicted | predicted | predicted | value after |
| Sample | Label | value | value | value | model fusion |
| BD-QCS-0207 | R | 0.902176582 | 0.583189646 | 0.61114869 | 0.698838306 |
| BD-YM-0503 | R | 0.743688313 | 0.622960578 | 0.699806401 | 0.688818431 |
| BD-SQ-0308 | NR | 0.797154387 | 0.273892945 | 0.384942178 | 0.485329837 |
| BD-HFS-0502 | R | 0.850361994 | 0.694019384 | 0.602268301 | 0.715549893 |
| BD-HLT-0605 | NR | 0.279250875 | 0.48845359 | 0.351676296 | 0.37312692 |
| BD-LZH-0301 | NR | 0.004627377 | 0.217784173 | 0.202440556 | 0.141617369 |
| BD-LBZ-0606 | R | 0.478354322 | 0.566531146 | 0.496470119 | 0.513785196 |
| BD-LJZ-0323 | R | 0.79682477 | 0.539020988 | 0.51356324 | 0.616469666 |
| BD-LRH-0523 | NR | 0.052163806 | 0.429432596 | 0.390800184 | 0.290798862 |
| BD-LLY-0530 | R | 0.560340895 | 0.562623225 | 0.526009328 | 0.549657816 |
| BD-LL-0403 | R | 0.874943417 | 0.686775463 | 0.632032379 | 0.731250419 |
| BD-WXJ-0412 | NR | 0.378518035 | 0.555143221 | 0.512520588 | 0.482060615 |
| BD-YMC-0213 | NR | 0.102155409 | 0.330534144 | 0.396371164 | 0.276353572 |
| BD-ZBL-0228 | NR | 0.99655608 | 0.23642188 | 0.30056981 | 0.51118259 |
| BD-ZXB-0326 | R | 0.761785749 | 0.588766354 | 0.66678056 | 0.672444221 |
| BD-ZZC-0428 | NR | 0.211648864 | 0.386474909 | 0.468036184 | 0.355386653 |
| BD-ZCW-0529 | NR | 0.170727948 | 0.353145515 | 0.350857871 | 0.291577112 |
| BD-ZQA-0524 | R | 0.673906679 | 0.617317301 | 0.617147662 | 0.636123881 |
| BD-ZLY-0604 | R | 0.63469881 | 0.555748818 | 0.579714156 | 0.590053928 |
| BD-PJL-0523 | R | 0.962658047 | 0.753885344 | 0.760669877 | 0.825737756 |
| BD-XBQ-0305 | NR | 0.670094683 | 0.481537488 | 0.389665409 | 0.51376586 |
| BD-LY-0604 | NR | 0.39482287 | 0.546709016 | 0.480988159 | 0.474173348 |
| BD-LSW-0314 | NR | 0.414030357 | 0.343745807 | 0.384499649 | 0.380758605 |
| BD-LYX-0606 | R | 0.84038549 | 0.703003809 | 0.663916042 | 0.735768447 |
| BD-LQR-0426 | R | 0.599522573 | 0.549899346 | 0.634684313 | 0.594702077 |
| BD-LDG-0606 | R | 0.689663826 | 0.622673486 | 0.589758132 | 0.634031815 |
| BD-DK-0307 | NR | 0.148947356 | 0.275750777 | 0.259992099 | 0.228230077 |
| BD-YZQ-0201 | R | 0.813329546 | 0.687548557 | 0.674427706 | 0.725101937 |
| BD-KL-0522 | R | 0.957900303 | 0.880399744 | 0.811687217 | 0.883329088 |
| BD-DCY-0308 | R | 0.841003768 | 0.43230092 | 0.547013873 | 0.606772853 |
| BD-SYJ-0316 | R | 0.435832045 | 0.545226809 | 0.491774069 | 0.490944307 |
| BD-JSZ-0427 | R | 0.810814583 | 0.646847853 | 0.71262007 | 0.723427502 |
| BD-WQL-0308 | R | 0.846052805 | 0.57196801 | 0.650467472 | 0.689496095 |
| BD-WJC-0522 | R | 0.880164403 | 0.614768088 | 0.600765939 | 0.698566143 |
| BD-WJC-0524 | R | 0.561728736 | 0.568899556 | 0.538119122 | 0.556249138 |
| BD-WJ-0322 | R | 0.817344939 | 0.666575032 | 0.530742659 | 0.67155421 |
| BD-SYC-0411 | R | 0.828118718 | 0.652859708 | 0.711182279 | 0.730720235 |
| BD-QXY-0212 | R | 0.690673251 | 0.661321173 | 0.632650576 | 0.661548333 |
| BD-ZML-0207 | NR | 2.60E−08 | 0.302460554 | 0.421722309 | 0.241394296 |
| BD-FGL-0209 | NR | 0.203420838 | 0.417643495 | 0.481272894 | 0.367445743 |
| BD-DXZ-0601 | R | 0.77978748 | 0.692881076 | 0.624876277 | 0.699181611 |
| BD-LMR-0315 | NR | 0.684290333 | 0.451202835 | 0.051264919 | 0.395586029 |
| BD-ZWB-0326 | R | 0.952440811 | 0.794287943 | 0.709529844 | 0.818752866 |
| BD-LJD-0426 | NR | 0.284715437 | 0.491218701 | 0.535395801 | 0.43710998 |
| BD-SCL-0409 | NR | 0.525732031 | 0.55951948 | 0.511541843 | 0.532264451 |
| BD-GFC-0419 | NR | 0.471015672 | 0.496659943 | 0.462357828 | 0.476677814 |
| BD-YJS-0606 | R | 0.694165523 | 0.607029266 | 0.609483953 | 0.636892914 |
| BD-CJR-0607 | R | 0.751969053 | 0.587941787 | 0.679213816 | 0.673041552 |
| BD-RXY-0307 | R | 0.250656227 | 0.476684514 | 0.453447199 | 0.39359598 |
| BD-LJS-0605 | R | 0.657495908 | 0.602818749 | 0.581082986 | 0.613799214 |

The AUC (Area Under Curve) of the three models used in the training set were all above 98%, and the AUC of the models in the test set were 76%, 90%, and 96% respectively, see Table 8.

TABLE 8
Model prediction results AUC

| Model | AUC in the training set | AUC in the test set |
|---|---|---|
| 1 | 99.5% | 76.67% |
| 2 | 98.9% | 90.0% |
| 3 | 98.2% | 96.1% |

Subsequently, the average of the predicted values according to the three models for each sample was used as the predicted value of the fusion model. 50 samples were predicted with the fusion model, and the resulting confusion matrix was shown in Table 9 below.

TABLE 9
Confusion matrix predicted by the fusion model for 50 samples

| | | Reference Value | |
|---|---|---|---|
| Confusion Matrix | | NR | R |
| Predicted Value | NR | 16 | 2 |
| | R | 3 | 29 |

Overall, the accuracy of the model was 90%, the sensitivity was 93.55%, and the specificity was up to 84.21%.

### Example 3. Prediction of Responsiveness Using the Presence and Abundance Information of Bacteria

In addition, the presence and abundance information of 15 bacterial genera as shown in Table 10 were used as features and their weight values were calculated. Among them, 7 genera (Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Ruminococcaceae Hydrogenoanaerobacterium and Desulfovibrionaceae Mailhella) were the same as those used in Example 2, and the other 8 genera (Burkholderiaceae Sutterella, Ruminococcaceae Oscillibacter, Ruminococcaceae Anaerofilum, Veillonellaceae Allisonella, Lachnospiraceae Lachnospiraceae_UCG-010, Erysipelotrichaceae Erysipelatoclostridium, Anaplasmataceae Wolbachia and Ruminococcaceae Butyricicoccus) were different from those used in Example 2.

TABLE 10
Summary of model variables and parameters

| | | Model 1 | Model 2 | Model 3 |
|---|---|---|---|---|
| j | Feature | Weight | Weight | Weight |
| | Intercept | 0.002178512 | 0.01472362 | 0.01631643 |
| 1 | Lachnospiraceae | 0 | 0.36762222 | 0.17078207 |
| | Lachnoclostridium | | | |
| 2 | Fusobacteriaceae | 0.225235336 | 0.42000227 | 0.35571732 |
| | Fusobacterium | | | |
| 3 | Erysipelotrichaceae | 0 | −0.3883258 | −0.1550203 |
| | Solobacterium | | | |
| 4 | Pasteurellaceae | −0.026693418 | −0.1070291 | −0.282037 |
| | Aggregatibacter | | | |
| 5 | Ruminococcaceae | −0.396090873 | −0.3707492 | −0.018458 |
| | Acetanaerobacterium | | | |
| 6 | Ruminococcaceae | 0.049490906 | −0.3740926 | −0.0008381 |
| | Hydrogenoanaerobacterium | | | |
| 7 | Desulfovibrionaceae | −0.277942592 | −0.4505819 | −0.2212571 |
| | Mailhella | | | |
| 8 | Ruminococcaceae | 0.002818753 | 0.46454505 | 0 |
| | Butyricicoccus | | | |
| 9 | Burkholderiaceae | 0 | −0.2223776 | −0.2309067 |
| | Sutterella | | | |
| 10 | Ruminococcaceae | −0.004572036 | −0.0610842 | 0 |
| | Oscillibacter | | | |
| 11 | Ruminococcaceae | 0 | 0 | 0 |
| | Anaerofilum | | | |
| 12 | Veillonellaceae | 0.364929071 | 0.4738331 | 0.35183279 |
| | Allisonella | | | |
| 13 | Anaplasmataceae | 0 | −0.0479556 | 0 |
| | Wolbachia | | | |
| 14 | Lachnospiraceae | 0 | 0.31289009 | 0 |
| | Lachnospiraceae_UCG-010 | | | |
| 15 | Erysipelotrichaceae | −0.260358078 | −0.1108514 | 0 |
| | Erysipelatoclostridium | | | |

Note:
Each parameter in the model came from the training set data. The model was trained and constructed through the training of the training set data, and used to predict the test set data.

The specific results calculated by using the features above and the formulae shown in Example 1 were shown in Table 11 below.

TABLE 11
Model prediction results

| | | Model 1 | Model 2 | Model 3 | Predicted |
|---|---|---|---|---|---|
| | | Predicted | Predicted | Predicted | value after |
| Sample | Label | value | value | value | model fusion |
| BD-QCS-0207 | R | 0.79482444 | 0.809027357 | 0.620203769 | 0.741351855 |
| BD-YM-0503 | R | 0.856088818 | 0.755046812 | 0.766314808 | 0.792483479 |
| BD-SQ-0308 | NR | 0.744814159 | 0.102190086 | 0.478526965 | 0.441843737 |
| BD-HFS-0502 | R | 0.496575851 | 0.648769941 | 0.533921365 | 0.559755719 |
| BD-HLT-0605 | NR | 0.495233432 | 0.556453371 | 0.453058168 | 0.501581657 |
| BD-LZH-0301 | NR | 0.575788455 | 0.224339387 | 0.360190718 | 0.386772853 |
| BD-LBZ-0606 | R | 0.540089423 | 0.790120064 | 0.56652499 | 0.632244826 |
| BD-LJZ-0323 | R | 0.530268035 | 0.620030301 | 0.46330367 | 0.537867335 |
| BD-LRH-0523 | NR | 0.533535775 | 0.589982452 | 0.352029374 | 0.4918492 |
| BD-LLY-0530 | R | 0.526202399 | 0.847699355 | 0.537786399 | 0.637229384 |
| BD-LL-0403 | R | 0.690296947 | 0.904253928 | 0.705126139 | 0.766559004 |
| BD-WXJ-0412 | NR | 0.494672545 | 0.35453599 | 0.389220261 | 0.412809599 |
| BD-YMC-0213 | NR | 0.230669834 | 0.198223635 | 0.416784274 | 0.281892581 |
| BD-ZBL-0228 | NR | 0.766815045 | 0.021097786 | 0.339968817 | 0.375960549 |
| BD-ZXB-0326 | R | 0.503055927 | 0.321162301 | 0.398785382 | 0.40766787 |
| BD-ZZC-0428 | NR | 0.465395626 | 0.139889395 | 0.244121023 | 0.283135348 |
| BD-ZCW-0529 | NR | 0.262473634 | 0.19722574 | 0.51418183 | 0.324627068 |
| BD-ZQA-0524 | R | 0.730023539 | 0.837051125 | 0.694577282 | 0.753883982 |
| BD-ZLY-0604 | R | 0.789961857 | 0.846906846 | 0.744670734 | 0.793846479 |
| BD-PJL-0523 | R | 0.827397064 | 0.891305728 | 0.761238995 | 0.826647262 |
| BD-XBQ-0305 | NR | 0.416308607 | 0.467981349 | 0.427247234 | 0.437179063 |
| BD-LY-0604 | NR | 0.507203347 | 0.773556993 | 0.537123475 | 0.605961272 |
| BD-LSW-0314 | NR | 0.522073937 | 0.279631555 | 0.301253489 | 0.367652993 |
| BD-LYX-0606 | R | 0.495652863 | 0.745393685 | 0.661962815 | 0.634336455 |
| BD-LQR-0426 | R | 0.805824115 | 0.594900121 | 0.609618107 | 0.670114115 |
| BD-LDG-0606 | R | 0.66171937 | 0.866847697 | 0.624709009 | 0.717758692 |
| BD-DK-0307 | NR | 0.344500274 | 0.142648075 | 0.281673084 | 0.256273811 |
| BD-YZQ-0201 | R | 0.564279541 | 0.826547083 | 0.490128226 | 0.62698495 |
| BD-KL-0522 | R | 0.804352627 | 0.975530932 | 0.853227976 | 0.877703845 |
| BD-DCY-0308 | R | 0.616212711 | 0.564512241 | 0.445318397 | 0.54201445 |
| BD-SYJ-0316 | R | 0.666653523 | 0.840477759 | 0.611778586 | 0.706303289 |
| BD-JSZ-0427 | R | 0.900529701 | 0.936579202 | 0.840898469 | 0.892669124 |
| BD-WQL-0308 | R | 0.578065845 | 0.580585424 | 0.610241681 | 0.589630983 |
| BD-WJC-0522 | R | 0.668490194 | 0.66226422 | 0.605540003 | 0.645431472 |
| BD-WJC-0524 | R | 0.555959359 | 0.725069108 | 0.527534238 | 0.602854235 |
| BD-WJ-0322 | R | 0.51635015 | 0.707696151 | 0.54614993 | 0.59006541 |
| BD-SYC-0411 | R | 0.514506082 | 0.764282478 | 0.600395471 | 0.626394677 |
| BD-QXY-0212 | R | 0.636351028 | 0.902985389 | 0.680645731 | 0.739994049 |
| BD-ZML-0207 | NR | 0.53003365 | 0.133355563 | 0.43913996 | 0.367509725 |
| BD-FGL-0209 | NR | 0.277795812 | 0.201915192 | 0.292826743 | 0.257512582 |
| BD-DXZ-0601 | R | 0.759167143 | 0.941628566 | 0.702653205 | 0.801149638 |
| BD-LMR-0315 | NR | 0.493877445 | 0.381555749 | 0.448473763 | 0.441302319 |
| BD-ZWB-0326 | R | 0.630787377 | 0.928405708 | 0.637887643 | 0.732360242 |
| BD-LJD-0426 | NR | 0.363241572 | 0.279173212 | 0.455010417 | 0.3658084 |
| BD-SCL-0409 | NR | 0.493573243 | 0.412200593 | 0.438485966 | 0.448086601 |
| BD-GFC-0419 | NR | 0.56975557 | 0.344117476 | 0.523863752 | 0.479245599 |
| BD-YJS-0606 | R | 0.495691858 | 0.465748735 | 0.385970649 | 0.449137081 |
| BD-CJR-0607 | R | 0.495860406 | 0.380766755 | 0.480027053 | 0.452218071 |
| BD-RXY-0307 | R | 0.500351112 | 0.556462059 | 0.484521724 | 0.513778298 |
| BD-LJS-0605 | R | 0.498552316 | 0.758914052 | 0.51733821 | 0.591601526 |

The predicted AUC values obtained using the above models and features and the confusion matrix predicted by the fusion model for 50 samples were shown in Tables 12 and 13.

TABLE 12
Model prediction results AUC

| | AUC in the | AUC in the |
|---|---|---|
| Model | training set | test set |
| 1 | 98.2% | 70.0% |
| 2 | 98.0% | 85.0% |
| 3 | 99.0% | 80.5% |

TABLE 13
Confusion matrix predicted by the fusion model for 50 samples

| | | Reference Value | |
|---|---|---|---|
| Confusion Matrix | | NR | R |
| Predicted Value | NR | 17 | 3 |
| | R | 2 | 28 |

Overall, the accuracy of the model was 90%, the sensitivity was 90.32%, and the specificity was up to 89.47%.

### Example 4. Prediction of Responsiveness Using the Presence and Abundance Information of Bacteria and the Patient's Allergy History

In addition, a model was constructed by selecting the patient's allergy history as one of the features and tested. Table 14 showed the used 14 bacterial genera and allergy history feature and weight values thereof.

TABLE 14
Summary of model variables and parameters

| | | Model 1 | Model 2 | Model 3 |
|---|---|---|---|---|
| | Variable | Weight | Weight | Weight |
| | Intercept | −0.007561151 | −0.02528504 | 0.035581174 |
| 1 | Lachnospiraceae | | | |
| | Lachnoclostridium | 0.269474217 | 0.114034718 | 0.258960313 |
| 2 | Fusobacteriaceae | 0.186344512 | 0.586043283 | 0.357814481 |
| | Fusobacterium | | | |
| 3 | Erysipelotrichaceae | −0.2170160959 | −0.498012396 | −0.317005109 |
| | Solobacterium | | | |
| 4 | Pasteurellaceae | −0.274545153 | −0.594097515 | −0.471015721 |
| | Aggregatibacter | | | |
| 5 | Ruminococcaceae | −0.260029833 | −0.482093741 | −0.55053872 |
| | Acetanaerobacterium | | | |
| 6 | Ruminococcaceae | −0.232073012 | −0.247073887 | −0.256377561 |
| | Hydrogenoanaerobacterium | | | |
| 7 | Desulfovibrionaceae | −0.295037845 | 0 | 0 |
| | Mailhella | | | |
| 8 | allergy history | 0.21318852 | 0.274294686 | 0.460397357 |
| 9 | Lachnospiraceae | −0.115359138 | −0.039416861 | −0.07425522 |
| | Coprococcus 2 | | | |
| 10 | Barnesiellaceae | −0.164532394 | −0.275271096 | −0.786574283 |
| | Barnesiella | | | |
| 11 | Prevotellaceae | −0.071830645 | −0.220218311 | −0.461396594 |
| | Prevotellaceae UCG-001 | | | |
| 12 | Erysipelotrichaceae | −0.149979281 | −0.702539539 | −0.056363688 |
| | Erysipelotrichaceae UCG-003 | | | |
| 13 | Ruminococcaceae | −0.196842716 | −0.26074899 | −0.260425181 |
| | Anaerotruncus | | | |
| 14 | Erysipelotrichaceae | −0.13582121 | −0.382900867 | −0.157556778 |
| | Faecalitalea | | | |
| 15 | Ruminococcaceae | −0.167621661 | −0.190137792 | −0.340468661 |
| | Ruminococcaceae UCG-008 | | | |

Note:
Each parameter in the model came from the training set data. The model was trained and constructed through the training of the training set data, and used to predict the test set data.

The specific results calculated by using the features above and the formulae shown in Example 1 were shown in Table 15 below.

TABLE 15
Model prediction results

| | | Model 1 | Model 2 | Model 3 | Predicted |
|---|---|---|---|---|---|
| | | Predicted | Predicted | Predicted | value after |
| Sample | Label | value | value | value | model fusion |
| BD-QCS-0207 | R | 0.609021619 | 0.798462688 | 0.775182947 | 0.72755575 |
| BD-YM-0503 | R | 0.723672142 | 0.824247001 | 0.831903485 | 0.79327421 |
| BD-SQ-0308 | NR | 0.078183058 | 0.199931635 | 0.320440182 | 0.19951829 |
| BD-HFS-0502 | R | 0.791833542 | 0.821722382 | 0.947179855 | 0.85357859 |
| BD-HLT-0605 | NR | 0.50224215 | 0.452279088 | 0.240260632 | 0.39826062 |
| BD-LZH-0301 | NR | 0.078546989 | 0.028648466 | 0.039239531 | 0.04881166 |
| BD-LBZ-0606 | R | 0.621593685 | 0.633297544 | 0.500852376 | 0.58524787 |
| BD-LJZ-0323 | R | 0.543752237 | 0.749109128 | 0.591949403 | 0.62827026 |
| BD-LRH-0523 | NR | 0.372143116 | 0.094846703 | 0.138988477 | 0.20199277 |
| BD-LLY-0530 | R | 0.559174503 | 0.55390302 | 0.618871913 | 0.57731648 |
| BD-LL-0403 | R | 0.764058316 | 0.834341235 | 0.851269374 | 0.81655631 |
| BD-WXJ-0412 | NR | 0.620772133 | 0.556429242 | 0.412724636 | 0.52997534 |
| BD-YMC-0213 | NR | 0.256180978 | 0.12432235 | 0.169787332 | 0.18343022 |
| BD-ZBL-0228 | NR | 0.018877655 | 0.248109482 | 0.086128668 | 0.11770527 |
| BD-ZXB-0326 | R | 0.693812383 | 0.834514297 | 0.815125138 | 0.78115061 |
| BD-ZZC-0428 | NR | 0.372053185 | 0.234482016 | 0.194115571 | 0.26688359 |
| BD-ZCW-0529 | NR | 0.271536919 | 0.249417402 | 0.092762829 | 0.20457238 |
| BD-ZQA-0524 | R | 0.715953468 | 0.722417536 | 0.723031337 | 0.72046745 |
| BD-ZLY-0604 | R | 0.759891778 | 0.902740894 | 0.9111857 | 0.85793946 |
| BD-PJL-0523 | R | 0.789552664 | 0.938514205 | 0.879989698 | 0.86935219 |
| BD-XBQ-0305 | NR | 0.288261331 | 0.164250327 | 0.210127901 | 0.22087985 |
| BD-LY-0604 | NR | 0.481069816 | 0.547083275 | 0.253647832 | 0.42726697 |
| BD-LSW-0314 | NR | 0.279223547 | 0.194494104 | 0.328938066 | 0.26755191 |
| BD-LYX-0606 | R | 0.802225403 | 0.774739625 | 0.814659568 | 0.7972082 |
| BD-LQR-0426 | R | 0.643438703 | 0.777932123 | 0.683712775 | 0.70169453 |
| BD-LDG-0606 | R | 0.693337352 | 0.709470256 | 0.679770754 | 0.69419279 |
| BD-DK-0307 | NR | 0.225355766 | 0.476656679 | 0.247342454 | 0.31645163 |
| BD-YZQ-0201 | R | 0.717381389 | 0.713383717 | 0.795486514 | 0.74208387 |
| BD-KL-0522 | R | 0.93330106 | 0.925890091 | 0.96939271 | 0.94286129 |
| BD-DCY-0308 | R | 0.373999774 | 0.533413673 | 0.574780688 | 0.49406471 |
| BD-SYJ-0316 | R | 0.67956626 | 0.761552639 | 0.764735673 | 0.73528486 |
| BD-JSZ-0427 | R | 0.759048509 | 0.844677441 | 0.859301353 | 0.8210091 |
| BD-WQL-0308 | R | 0.628134672 | 0.849928404 | 0.798195359 | 0.75875281 |
| BD-WJC-0522 | R | 0.61190109 | 0.799723342 | 0.775406538 | 0.72901032 |
| BD-WJC-0524 | R | 0.714902696 | 0.799878024 | 0.799229997 | 0.77133691 |
| BD-WJ-0322 | R | 0.598895139 | 0.61771032 | 0.572471078 | 0.59635885 |
| BD-SYC-0411 | R | 0.71545707 | 0.819959966 | 0.836486493 | 0.79063451 |
| BD-QXY-0212 | R | 0.844730666 | 0.925121932 | 0.924873276 | 0.89824196 |
| BD-ZML-0207 | NR | 0.280778649 | 0.034565708 | 0.191442921 | 0.16892909 |
| BD-FGL-0209 | NR | 0.403784564 | 0.708015423 | 0.833099902 | 0.64829996 |
| BD-DXZ-0601 | R | 0.760442031 | 0.831111129 | 0.670492723 | 0.75401529 |
| BD-LMR-0315 | NR | 0.340519098 | 0.172130031 | 0.000866754 | 0.17117196 |
| BD-ZWB-0326 | R | 0.682013668 | 0.841589773 | 0.784135235 | 0.76924623 |
| BD-LJD-0426 | NR | 0.460174806 | 0.232868616 | 0.712146373 | 0.4683966 |
| BD-SCL-0409 | NR | 0.573829193 | 0.643199879 | 0.434680809 | 0.55056996 |
| BD-GFC-0419 | NR | 0.223603137 | 0.255660514 | 0.137803776 | 0.20568914 |
| BD-YJS-0606 | R | 0.629623838 | 0.717780989 | 0.628131639 | 0.65851216 |
| BD-CJR-0607 | R | 0.702936628 | 0.83220844 | 0.821481788 | 0.78554228 |
| BD-RXY-0307 | R | 0.474833061 | 0.321151442 | 0.526701688 | 0.4408954 |
| BD-LJS-0605 | R | 0.697640827 | 0.740667914 | 0.644777889 | 0.69436221 |

The predicted AUC values and the confusion matrix obtained using the above models and features were shown in Tables 16 and 17.

TABLE 16
Model prediction results AUC

| | AUC in the | AUC in the |
|---|---|---|
| Model | training set | test set |
| 1 | 99.5% | 95.0% |
| 2 | 99.5% | 90.0% |
| 3 | 100% | 94.8% |

TABLE 17
Confusion matrix predicted by the fusion model for 50 samples

| | | Reference Value | |
|---|---|---|---|
| Confusion Matrix | | NR | R |
| Predicted Value | NR | 16 | 2 |
| | R | 3 | 29 |

Overall, the accuracy of the model was 90%, the sensitivity was 93.55%, and the specificity was up to 84.21%.

SEQUENCE LISTING
SEQ ID NO: 1
GTAAAGGGAGCGTAGACGGTAAAGCAAGTCTGAAGTGAAAGCCCGGGGCTC
AACCCCGGGACTGCTTTGGAAACTGTTTAACTAGAGTGCTGGAGAGGTAAG
CGGAATTCCTAGTGTAGCGGTGAAATGCGTAGATATTAGGAGGAACACCAG
TGGCGAAGGCGGCTTACTGGACAGTAACTGACGTTGAGGCTCGAAAGCGTG
GGGAGCAAACAGG
SEQ ID NO: 2
CGTAAAGCGCGTCTAGGCGGTTTGGTAAGTCTGATGTGAAAATGCGGGGCT
CAACTCCGTATTGCGTTGGAAACTGCCAAACTAGAGTACTGGAGAGGTGGG
CGGAACTACAAGTGTAGAGGTGAAATTCGTAGATATTTGTAGGAATGCCAA
TGGGGAAGCCAGCCCACTGGACAGATACTGACGCTAAAGCGCGAAAGCGTG
GGTAGCAAACAGG
SEQ ID NO: 3
CGTAAAGGGTGCGTAGGCGGCCTGTTAAGTAAGTGGTTAAATTGTTGGGCT
CAACCCAATCCAGCCACTTAAACTGGCAGGCTAGAGTATTGGAGAGGCAAG
TGGAATTCCATGTGTAGCGGTAAAATGCGTAGATATATGGAGGAACACCAG
TGGCGAAGGCGGCTTGCTAGCCAAAGACTGACGCTCATGCACGAAAGCGTG
GGGAGCAAATAGG
SEQ ID NO: 4
GTAAAGGGCACGCAGGCGGACTTTTAAGTGAGGTGTGAAATCCCCGGGCTT
AACCTGGGAATTGCATTTCAGACTGGGGGTCTAGAGTACTTTAGGGAGGGG
TAGAATTCCACGTGTAGCGGTGAAATGCGTAGAGATGTGGAGGAATACCGA
AGGCGAAGGCAGCCCCTTGGGAATGTACTGACGCTCATGTGCGAAAGCGTG
GGGAGCAAACAGG
SEQ ID NO: 5
GTAAAGGGAGCGTAGGCGGTTTGGTAAGTTGAGTGTGAAATCTACCGGCTT
AACTGGTAGGCTGCGCTCAAAACTACCAAACTTGAGTGAAGTAGAGGCAGG
CGGAATTCCCGGTGTAGCGGTGGAATGCGTAGATATCGGGAGGAACACCAG
TGGCGAAGGCGGCCTGCTGGGCTTTTACTGACGCTGATGCTCGAAAGCATG
GGGAGCAAACAGG
SEQ ID NO: 6
TGTAAAGGGAGCGTAGGCGGGAAGACAAGTTGAATGTTAAATCTATCGGCT
CAACCGGTAGCCGCGTTCAAAACTGTTTTTCTTGAGTGAAGTAGAGGTTGG
CGGAATTCCTAGTGTAGCGGTGAAATGCGTAGATATTAGGAGGAACACCAG
TGGCGAAGGCGGCCAACTGGGCTTTTACTGACGCTGAGGCTCGAAAGCGTG
GGGAGCAAACAGG
SEQ ID NO: 7
GTAAAGCGCATGTAGGCCGTGTGGCAAGTTAGGGGTGAAATCCCAGGGCTC
AACCTTGGAACTGCCTCTAAAACTACCATGCTTGAGTGCGAGAGAGGATAG
CGGAATTCCAGGTGTAGGAGTGAAATCCGTAGATATCTGGAAGAACATCAG
TGGCGAAGGCGGCTATCTGGCTCGTAACTGACGCTGAGATGCGAAAGCGTG
GGTAGCAAACAGG
SEQ ID NO: 8
GTAAAGGGTGCGTAGGTGGTGAGACAAGTCTGAAGTGAAAATCCGGGGCTT
AACCCCGGAACTGCTTTGGAAACTGCCTGACTAGAGTACAGGAGAGGTAAG
TGGAATTCCTAGTGTAGCGGTGAAATGCGTAGATATTAGGAGGAACACCAG
TGGCGAAGGCGACTTACTGGACTGCTACTGACACTGAGGCACGAAAGCGTG
GGGAGCAAACAGG
SEQ ID NO: 9
TTAAAGGGTGCGTAGGCGGCACGCCAAGTCAGCGGTGAAATTTCCGGGCTC
AACCCGGACTGTGCCGTTGAAACTGGCGAGCTAGAGTGCACAAGAGGCAGG
CGGAATGCGTGGTGTAGCGGTGAAATGCATAGATATCACGCAGAACCCCGA
TTGCGAAGGCAGCCTGCTAGGGTGAAACAGACGCTGAGGCACGAAAGCGTG
GGTATCGAACAGG
SEQ ID NO: 10
TTAAAGGGAGCGCAGGCGGCCTTTTAAGCGTGACGTGAAATGCCGGGGCTC
AACCTTGGAATTGCGTCGCGAACTGGCGGGCTTGAGTACGCTCGAGGCAGG
CGGAATTCGTGGTGTAGCGGTGAAATGCTTAGATATCACGAGGAACCCCGA
TTGCGAAGGCAGCCTGCCGGGGTGTTACTGACGCTCATGCTCGAAGGTGCG
GGTATCGAACAGG
SEQ ID NO: 11
TGTAAAGGGAGCGTAGGCGGGATGGCAAGTTGGATGTTTAAACTAACGGCT
CAACTGTTAGGTGCATCCAAAACTGCTGTTCTTGAGTGAAGTAGAGGCAGG
CGGAATTCCTAGTGTAGCGGTGAAATGCGTAGATATTAGGAGGAACACCAG
TGGCGAAGGCGGCCTGCTGGGCTTTAACTGACGCTGAGGCTCGAAAGCGTG
GGGAGCAAACAGG
SEQ ID NO: 12
CGTAAAGAGGGAGCAGGCGGCACTAAGGGTCTGTGGTGAAAGATCGAAGCT
TAACTTCGGTAAGCCATGGAAACCGTAGAGCTAGAGTGTGTGAGAGGATCG
TGGAATTCCATGTGTAGCGGTGAAATGCGTAGATATCACGAAGAACTCCGA
TTGCGAAGGCAGCCTGCTAAGCTGCAACTGACATTGAGGCTCGAAAGTGTG
GGTATCAAACAGG
SEQ ID NO: 13
CGTAAAGGGTGCGTAGGTGGTGCATTAAGTCTGAAGTAAAAGCCAGCAGCT
CAACTGCTGTAAGCTTTGGAAACTGGTGTACTAGAGTGCAGGAGAGGGCGA
TGGAATTCCATGTGTAGCGGTAAAATGCGTAGATATATGGAGGAACACCAG
TGGCGAAGGCGGTCGCCTGGCCTGTAACTGACACTGAGGCACGAAAGCGTG
GGGAGCAAATAGG
SEQ ID NO: 14
GTAAAGGGAGCGTAGGCGGCGACGCAAGTCAGAAGTGAAAGCCCGGGGCTC
AACTCCGGGACTGCTTTTGAAACTGCGTTGCTAGATTGCGGGAGAGGCAAG
TGGAATTCCTAGTGTAGCGGTGAAATGCGTAGATATTAGGAGGAACACCAG
TGGCGAAGGCGGCTTGCTGGACCGTGAATGACGCTGAGGCTCGAAAGCGTG
GGGAGCAAACAGG
SEQ ID NO: 15
GTAAAGGGCGAGTAGGCGGGTCGGCAAGTTGGGAGTGAAATGTCGGGGCTT
AACCCCGGAACTGCTTCCAAAACTGTTGATCTTGAGTGATGGAGAGGCAGG
CGGAATTCCCAGTGTAGCGGTGAAATGCGTAGATATTGGGAGGAACACCAG
TGGCGAAGGCGGCCTGCTGGACATTAACTGACGCTGAGGAGCGAAAGCGTG
GGGAGCAAACAGG
SEQ ID NO: 16
GTAAAGGGTGAGTAGGCGGCATGGTAAGTTAGATGTGAAAGCCCGGGGCTT
AACCCCGGGATTGCATTTAAAACTATCAAGCTCGAGTTCAGGAGAGGTAAG
CGGAATTCCTAGTGTAGCGGTGAAATGCGTAGATATTAGGAAGAACACCGG
TGGCGAAGGCGGCTTACTGGACTGATACTGACGCTGAGGCACGAAAGCGTG
GGGAGCAAACAGG
SEQ ID NO: 17
GTAAAGGGCGCGCAGGCGGGCCGGTAAGTTGGAAGTGAAATCTATGGGCTT
AACCCATAAACTGCTTTCAAAACTGCTGGTCTTGAGTGATGGAGAGGCAGG
CGGAATTCCGTGTGTAGCGGTGAAATGCGTAGATATACGGAGGAACACCAG
TGGCGAAGGCGGCCTGCTGGACATTAACTGACGCTGAGGCGCGAAAGCGTG
GGGAGCAAACAGG
SEQ ID NO: 18
GTAAAGGGTGCGCAGGCGGCTGTGCAAGACAGATGTGAAATCCCCGGGCTT
AACCTGGGAACTGCATTTGTGACTGCACGGCTAGAGTTTGTCAGAGGAGGG
TGGAATTCCGCGTGTAGCAGTGAAATGCGTAGATATGCGGAAGAACACCAA
TGGCGAAGGCAGCCCTCTGGGACATGACTGACGCTCATGCACGAAAGCGTG
GGGAGCAAACAGG
SEQ ID NO: 19
GTAAAGGGTGCGTAGGTGGCCATGTAAGTTAGGTGTGAAAGACCGGGGCTT
AACCCCGGGGCGGCACTTAAAACTGTGTGGCTTGAGTACAGGAGAGGGAAG
TGGAATTCCTAGTGTAGCGGTGAAATGCGTAGATATTAGGAGGAACACCAG
TGGCGAAGGCGACTTTCTGGACTGTAACTGACACTGAGGCACGAAAGCGTG
GGGAGCAAACAGG
SEQ ID NO: 20
GTAAAGGGCGTGTAGCCGGGTCGGCAAGTCAGATGTGAAATCCACGGGCTT
AACCCGTGAACTGCATTTGAAACTGCTGATCTTGAGTGTCGGAGAGGTAAT
CGGAATTCCTAGTGTAGCGGTGAAATGCGTAGATATTAGGAAGAACACCGG
TGGCGAAGGCGGATTACTGGACGATAACTGACGGTGAGGCGCGAAAGCGTG
GGGAGCAAACAGG
SEQ ID NO: 21
GTAAAGGGCGCGCAGGCGGCTGTGTAAGTCTGTCTAGAAAGTGCGGGGCTA
AACCCCGTGAGAGGATGGAAACTGGACAGCTGAGAGTGTCGGAGAGGAAAG
CGGAATTCCTAGTGTAGCGGTGAAATGCGTAGATATTAGGAGGAACACCGG
TGGCGAAAGCGGCTTTCTGGACGACAACTGACGCTGAGGCGCGAAAGCCAG
GGGAGCAAACGGG
SEQ ID NO: 22
TGTAAAGGGAGCGCAGGCGGAGCTGTAAGTTGGGCGTCAAATCTACGGGCT
TAACCCGTATCCGCGCTCAAAACTGTGGCTCTTGAGTAGTGCAGAGGTAGG
TGGAATTCCCGGTGTAGCGGTGGAATGCGTAGATATCGGGAGGAACACCAG
TGGCGAAGGCGGCCTACTGGGCACCAACTGACGCTGAGGCTCGAAAGTATG
GGTAGCAAACAGG
SEQ ID NO: 23
TGTAAAGGGAGCGTAGGCGGGTACGCAAGTTGAATGTGAAAACTAACGGCT
CAACCGATAGTTGCGTTCAAAACTGCGGATCTTGAGTGAAGTAGAGGCAGG
CGGAATTCCTAGTGTAGCGGTAAAATGCGTAGATATTAGGAGGAACACCAG
TGGCGAAGGCGGCCTGCTGGGCTTTAACTGACGCTGAGGCTCGAAAGTGTG
GGGAGCAAACAGG
SEQ ID NO: 24
GTAAAGGGAGCGTAGACGGAATGGCAAGTCTGAAGTGAAATACCCGGGCTC
AACCTGGGAACTGCTTTGGAAACTGTTGTTCTAGAGTGTTGGAGAGGTAAG
TGGAATTCCTGGTGTAGCGGTGAAATGCGTAGATATCAGGAAGAACACCGG
AGGCGAAGGCGGCTTACTGGACAATAACTGACGTTGAGGCTCGAAAGCGTG
GGGATCAAACAGG
SEQ ID NO: 25
CGTAAAGCGCGCGCAGGCGGCCGTGCAAGTCCATCTTAAAAGCGTGGGGCT
TAACCCCATGAGGGGATGGAAACTGCATGGCTGGAGTGTCGGAGGGGAAAG
TGGAATTCCTAGTGTAGCGGTGAAATGCGTAGAGATTAGGAAGAACACCGG
TGGCGAAGGCGACTTTCTAGACGACAACTGACGCTGAGGCGCGAAAGCGTG
GGGAGCAAACAGG
SEQ ID NO: 26
GTAAAGCGCGCGCAGGCGGTCTCTTAAGTCTGATGTGAAAGCCCCCGGCTC
AACCGGGGAGGGTCATTGGAAACTGGGAGACTTGAGGACAGAAGAGGAGAG
TGGAATTCCAAGTGTAGCGGTGAAATGCGTAGATATTTGGAGGAACACCAG
TGGCGAAGGCGGCTCTCTGGTCTGTTACTGACGCTGAGGCGCGAAAGCGTG
GGGAGCAAACAGG
SEQ ID NO: 27
GTAAAGGGCGCGTAGGCTGATTAATAAGTTAAAAGTGAAATCCCGAGGCTT
AACCTTGGAATTGCTTTTAAAACTATTAATCTAGAGATTGAAAGAGGATAG
AGGAATTCCTGATGTAGAGGTAAAATTCGTAAATATTAGGAGGAACACCAG
TGGCGAAGGCGTCTATCTGGTTCAAATCTGACGCTGAGGCGCGAAGGCGTG
GGGAGCAAACAGG
SEQ ID NO: 28
GTAAAGAGCTCGTAGGCGGTATATTAAGTCAGATGTGAAATCCCTTGGCTT
AACCTAGGAACTGCATTTGAAACTGATAAACTAGAGTATCGTAGAGGGAGG
TAGAATTCTAGGTGTAGCGGTGAAATGCGTAGATATCTGGAGGAATACCTG
TGGCGAAAGCGACCTCCTAAACGAATACTGACGCTGAGGTGCGAAAGCGTG
GGGAGCAAACAGG
SEQ ID NO: 29
TAAAGGGTGAGTAGGCGGCATGGCAAGTAAGATGTGAAAGCCCGAGGCTTA
ACCTCGGGATTGCATTTTAAACTGCTAAGCTAGAGTACAGGAGAGGAAAGC
GGAATTCCTAGTGTAGCGGTGAAATGCGTAGATATTAGGAAGAACACCAGT
GGCGAAGGCGGCTTTCTGGACTGGAAACTGACGCTGAGGCACGAAAGCGTG
GGGAGCGAACAGG
SEQ ID NO: 30
GTAAAGCGTGTGTAGGCGGTTCGGAAAGAAAGATGTGAAATCCCAGGGCTC
AACCTTGGAACTGCATTTTTAACTGCCGAGCTAGAGTATGTCAGAGGGGGG
TAGAATTCCACGTGTAGCAGTGAAATGCGTAGATATGTGGAGGAATACCGA
TGGCGAAGGCAGCCCCCTGGGATAATACTGACGCTCAGACACGAAAGCGTG
GGGAGCAAACAGG
SEQ ID NO: 31
CGTAAAGAGGGAGCAGGCGGCGGCAGAGGTCTGTGGTGAAAGACTGAAGCT
TAACTTCAGTAAGCCATAGAAACCGGGCTGCTAGAGTGCAGGAGAGGATCG
TGGAATTCCATGTGTAGCGGTGAAATGCGTAGATATATGGAGGAACACCAG
TGGCGAAGGCGACGGTCTGGCCTGTAACTGACGCTCATTCCCGAAAGCGTG
GGGAGCAAACAGG
SEQ ID NO: 32
GTAAAGGGAGCGTAGACGGCTGTGTAAGTCTGAAGTGAAAGCCCGGGGCTC
AACCCCGGGACTGCTTTGGAAACTATGCAGCTAGAGTGTCGGAGAGGTAAG
TGGAATTCCCAGTGTAGCGGTGAAATGCGTAGATATTGGGAGGAACACCAG
TGGCGAAGGCGGCTTACTGGACGATGACTGACGTTGAGGCTCGAAAGCGTG
GGGAGCAAACAGG
SEQ ID NO: 33
GTAAAGCGCACGCAGGCGGTTGCCCAAGTCAGATGTGAAAGCCCCGGGCTT
AACCTGGGAACTGCATTTGAAACTGGGCGACTAGAGTATGAAAGAGGAAAG
CGGAATTTCCAGTGTAGCAGTGAAATGCGTAGATATTGGAAGGAACACCGA
TGGCGAAGGCAGCTTTCTGGGTCGATACTGACGCTCATGTGCGAAAGCGTG
GGGAGCAAACAGG

1. A method for identifying a responsiveness of a subject to immune checkpoint inhibitor therapy, comprising:
a) providing a sample comprising the gut microbiota of the subject;
b) detecting the presence and abundance information of microorganisms of one or more genera selected from the group consisting of genera listed in the following table in the sample:
Lachnospiraceae Lachnoclostridium
Fusobacteriaceae Fusobacterium
Erysipelotrichaceae Solobacterium
Pasteurellaceae Aggregatibacter
Ruminococcaceae Acetanaerobacterium
Ruminococcaceae Hydrogenoanaerobacterium
Desulfovibrionaceae Mailhella
Lachnospiraceae Coprococcus_2
Barnesiellaceae Barnesiella
Prevotellaceae Prevotellaceae_UCG-001
Ruminococcaceae Anaerotruncus
Erysipelotrichaceae Erysipelotrichaceae_UCG-003
Erysipelotrichaceae Faecalitalea
Lachnospiraceae GCA-900066575
Ruminococcaceae Ruminococcaceae_UCG-008
Lachnospiraceae Tyzzerella
Ruminococcaceae Butyricicoccus
Burkholderiaceae Sutterella
Christensenellaceae Catabacter
Ruminococcaceae Oscillibacter
Veillonellaceae Anaeroglobus
Ruminococcaceae Anaerofilum
Ruminococcaceae Candidatus_Soleaferrea
Lachnospiraceae Oribacterium
Veillonellaceae Allisonella
Listeriaceae Brochothrix
Anaplasmataceae Wolbachia
Enterobacteriaceae Buchnera
Lachnospiraceae Lachnospiraceae_UCG-010
Burkholderiaceae Alcaligenes
Erysipelotrichaceae Erystpelatoclostridium
Lachnospiraceae Coprococcus_3
Cardiobacteriaceae Cardiobacterium
c) identifying the subject's responsiveness to immune checkpoint inhibitor therapy through the presence and abundance information of the microorganisms of the one or more genera.

2. The method of claim 1, wherein the immune checkpoint inhibitor therapy is a PD-1 signaling pathway inhibitor.

3. The method of claim 2, wherein the PD-1 signaling pathway inhibitor is selected from the group consisting of a PD-1 inhibitor and a PD-L1 inhibitor.

4. The method of claim 1, wherein the subject has cancer.

5. The method of claim 4, wherein the cancer is a digestive tract cancer.

6. The method of claim 4, wherein the cancer is selected from the group consisting of an esophageal cancer, a gastric cancer, an ampullary cancer, a colorectal cancer, a sarcoidosis, a pancreatic cancer, a nasopharyngeal cancer, a neuroendocrine tumor, a melanoma, a non-small cell lung cancer, a liver cancer and a kidney cancer.

7. The method of claim 1, wherein the subject is receiving or preparing to receive the immune checkpoint inhibitor therapy.

8. The method of claim 1, wherein the sample is an intestinal tissue sample or a stool sample.

9. The method of claim 1, wherein the one or more genera includes at least one genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

10. The method of claim 9, wherein the one or more genera includes all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaero bacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea and Ruminococcaceae Ruminococcaceae_UCG-008.

11. The method of claim 9, wherein the one or more genera includes all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaero bacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

12. The method of claim 1, wherein the presence and abundance information of the microorganisms are detected by targeted sequencing analysis, metagenomic sequencing analysis, or qPCR (quantitative polymerase chain reaction) analysis.

13. The method of claim 12, wherein the targeted sequencing analysis is 16s rDNA sequencing analysis.

14. The method of claim 1, wherein the presence and abundance information of the microorganisms of the one or more genera are detected by detecting the presence and abundance information of a nucleotide sequence having at least 70% of sequence identity to a nucleotide sequence selected from the following table in the sample:

| | |
|---|---|
| Lachnospiraceae Lachnoclostridium | SEQ ID NO: 1 |
| Fusobacteriaceae Fusobacterium | SEQ ID NO: 2 |
| Erysipelotrichaceae Solobacterium | SEQ ID NO: 3 |
| Pasteurellaceae Aggregatibacter | SEQ ID NO: 4 |
| Ruminococcaceae Acetanaerobacterium | SEQ ID NO: 5 |
| Ruminococcaceae Hydrogenoanaerobacterium | SEQ ID NO: 6 |
| Desulfovibrionaceae Mailhella | SEQ ID NO: 7 |
| Lachnospiraceae Coprococcus_2 | SEQ ID NO: 8 |
| Barnesiellaceae Barnesiella | SEQ ID NO: 9 |
| Prevotellaceae Prevotellaceae_UCG-001 | SEQ ID NO: 10 |
| Ruminococcaceae Anaerotruncus | SEQ ID NO: 11 |
| Erysipelotrichaceae Erysipelotrichaceae_UCG-003 | SEQ ID NO: 12 |
| Erysipelotrichaceae Faecalitalea | SEQ ID NO: 13 |
| Lachnospiraceae GCA-900066575 | SEQ ID NO: 14 |
| Ruminococcaceae Ruminococcaceae_UCG-008 | SEQ ID NO: 15 |
| Lachnospiraceae Tyzzerella | SEQ ID NO: 16 |
| Ruminococcaceae Butyricicoccus | SEQ ID NO: 17 |
| Burkholderiaceae Sutterella | SEQ ID NO: 18 |
| Chri stens enellaceae Catabacter | SEQ ID NO: 19 |
| Ruminococcaceae Oscillibacter | SEQ ID NO: 20 |
| Veillonellaceae Anaeroglobus | SEQ ID NO: 21 |
| Ruminococcaceae Anaerofilum | SEQ ID NO: 22 |
| Ruminococcaceae Candidatus_Soleaferrea | SEQ ID NO: 23 |
| Lachnospiraceae Oribacterium | SEQ ID NO: 24 |
| Veillonellaceae Allisonella | SEQ ID NO: 25 |
| Listeriaceae Brochothrix | SEQ ID NO: 26 |
| Anaplasmataceae Wolbachia | SEQ ID NO: 27 |
| Enterobacteriaceae Buchnera | SEQ ID NO: 28 |
| Lachnospiraceae Lachnospiraceae_UCG-010 | SEQ ID NO: 29 |
| Burkholderiaceae Alcaligenes | SEQ ID NO: 30 |
| Erysipelotrichaceae Erysipelatoclostridium | SEQ ID NO: 31 |
| Lachnospiraceae Coprococcus_3 | SEQ ID NO: 32 |
| Cardiobacteriaceae Cardiobacterium | SEQ ID NO: 33 |

15. The method of claim 14, wherein the presence and abundance information of the microorganisms of the one or more genera are detected by detecting the presence and abundance information of a nucleotide sequence having at least 75% of sequence identity to a nucleotide sequence selected from the following table in the sample.

16. The method of claim 14, wherein the presence and abundance information of the microorganisms of the one or more genera are detected by detecting the presence and abundance information of a nucleotide sequence having at least 80% of sequence identity to a nucleotide sequence selected from the following table in the sample.

17. The method of claim 14, wherein the presence and abundance information of the microorganisms of the one or more genera are detected by detecting the presence and abundance information of a nucleotide sequence having at least 85% of sequence identity to a nucleotide sequence selected from the following table in the sample.

18. The method of claim 14, wherein the presence and abundance information of the microorganisms of the one or more genera are detected by detecting the presence and abundance information of a nucleotide sequence having at least 90% of sequence identity to a nucleotide sequence selected from the following table in the sample.

19. The method of claim 14, wherein the presence and abundance information of the microorganisms of the one or more genera are detected by detecting the presence and abundance information of a nucleotide sequence having at least 95% of sequence identity to a nucleotide sequence selected from the following table in the sample.

20. The method of claim 1, wherein in step c) the responsiveness of the subject to immune checkpoint inhibitor therapy is identified by a machine learning method.

21. The method of claim 20, wherein the machine learning method comprises a random forest model or a logistic regression model.

22. The method of claim 21, wherein the random forest model or logistic regression model further includes using the presence and abundance information of other types of microorganisms as a feature.

23. The method of claim 20 or 21, wherein the random forest model or logistic regression model further includes using the subject's allergy history as a feature.

24. The method of claim 1, wherein the subject is identified as responsive or non-responsive to the immune checkpoint inhibitor therapy.

25-50. (canceled)

51. A kit for identifying a responsiveness of a subject to immune checkpoint inhibitor therapy, the kit containing a detection reagent for detecting the presence and abundance information of microorganisms of one or more genera selected from the group consisting of genera listed in the following table in a sample comprising the gut microbiota of the subject:
Lachnospiraceae Lachnoclostridium
Fusobacteriaceae Fusobacterium
Erysipelotrichaceae Solobacterium
Pasteurellaceae Aggregatibacter
Ruminococcaceae Acetanaerobacterium
Ruminococcaceae Hydrogenoanaerobacterium
Desulfovibrionaceae Mailhella
Lachnospiraceae Coprococcus_2
Barnesiellaceae Barnesiella
Prevotellaceae Prevotellaceae_UCG-001
Ruminococcaceae Anaerotruncus
Erysipelotrichaceae Erysipelotrichaceae_UCG-003
Erysip elotrichaceae Faecalitalea
Lachnospiraceae GCA-900066575
Ruminococcaceae Ruminococcaceae_UCG-008
Lachnospiraceae Tyzzerella
Ruminococcaceae Butyricicoccus
Burkholderiaceae Sutterella
Christensenellaceae Catabacter
Ruminococcaceae Oscillibacter
Veillonellaceae Anaeroglobus
Ruminococcaceae Anaerofilum
Ruminococcaceae Candidatus_Soleaferrea
Lachnospiraceae Oribacterium
Veillonellaceae Allisonella
Listeriaceae Brochothrix
Anaplasmataceae Wolbachia
Enterobacteriaceae Buchnera
Lachnospiraceae Lachnospiraceae_UCG-010
Burkholderiaceae Alcaligenes
Erysipelotrichaceae Erysipelatoclostridium
Lachnospiraceae Coprococcus_3
Cardiobacteriaceae Cardiobacterium

52. The kit of claim 51, wherein the immune checkpoint inhibitor therapy is a PD-1 signaling pathway inhibitor.

53. The kit of claim 52, wherein the PD-1 signaling pathway inhibitor is selected from the group consisting of a PD-1 inhibitor and a PD-L1 inhibitor.

54. The kit of claim 51, wherein the subject has cancer.

55. The kit of claim 54, wherein the cancer is a digestive tract cancer.

56. The kit of claim 54, wherein the cancer is selected from the group consisting of an esophageal cancer, a gastric cancer, an ampullary cancer, a colorectal cancer, a sarcoidosis, a pancreatic cancer, a nasopharyngeal cancer, a neuroendocrine tumor, a melanoma, a non-small cell lung cancer, a liver cancer and a kidney cancer.

57. The kit of claim 51, wherein the subject is receiving or preparing to receive the immune checkpoint inhibitor therapy.

58. The kit of claim 51, wherein the sample is an intestinal tissue sample or a stool sample.

59. The kit of claim 51, wherein the one or more genera includes at least one, for example at least two, for example at least five genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaerobacterium, Desulfovibrionaceae Mailhella, Bamesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

60. The kit of claim 59, wherein the one or more genera includes all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaero bacterium, Desulfovibrionaceae Mailhella, Bamesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea and Ruminococcaceae Ruminococcaceae_UCG-008.

61. The kit of claim 59, wherein the one or more genera includes all genera selected from the group consisting of Lachnospiraceae Lachnoclostridium, Fusobacteriaceae Fusobacterium, Erysipelotrichaceae Solobacterium, Pasteurellaceae Aggregatibacter, Ruminococcaceae Acetanaerobacterium, Lachnospiraceae Coprococcus_2, Ruminococcaceae Hydrogenoanaero bacterium, Desulfovibrionaceae Mailhella, Barnesiellaceae Barnesiella, Prevotellaceae Prevotellaceae_UCG-001, Ruminococcaceae Anaerotruncus, Erysipelotrichaceae Erysipelotrichaceae_UCG-003, Erysipelotrichaceae Faecalitalea, Ruminococcaceae Ruminococcaceae_UCG-008 and Lachnospiraceae GCA-900066575.

62. The kit of claim 51, wherein the detection reagent is specific primers for the genomic DNA of the microorganisms of the one or more genera.

63. The kit of claim 62, wherein the primers are specific primers or qPCR primers for 16s rDNA of microorganisms of the one or more genera.

64. The kit of claim 62, wherein the presence and abundance information of microorganisms of the one or more genera is obtained by a PCR reaction using the primers and using the genomic DNA of the subject's gut microbiota as a template.

65. The kit of claim 51, wherein the presence and abundance information of the microorganisms of the one or more genera are detected by detecting the presence and abundance information of a nucleotide sequence having at least 70% of sequence identity to a nucleotide sequence selected from the following group or a fragment thereof in the sample:

| | |
|---|---|
| Lachnospiraceae Lachnoclostridium | SEQ ID NO: 1 |
| Fusobacteriaceae Fusobacterium | SEQ ID NO: 2 |
| Erysipelotrichaceae Solobacterium | SEQ ID NO: 3 |
| Pasteurellaceae Aggregatibacter | SEQ ID NO: 4 |
| Ruminococcaceae Acetanaerobacterium | SEQ ID NO: 5 |
| Ruminococcaceae Hydrogenoanaerobacterium | SEQ ID NO: 6 |
| Desulfovibrionaceae Mailhella | SEQ ID NO: 7 |
| Lachnospiraceae Coprococcus_2 | SEQ ID NO: 8 |
| Barnesiellaceae Barnesiella | SEQ ID NO: 9 |
| Prevotellaceae Prevotellaceae_UCG-001 | SEQ ID NO: 10 |
| Ruminococcaceae Anaerotruncus | SEQ ID NO: 11 |
| Erysipelotrichaceae Erysipelotrichaceae_UCG-003 | SEQ ID NO: 12 |
| Erysipelotrichaceae Faecalitalea | SEQ ID NO: 13 |
| Lachnospiraceae GCA-900066575 | SEQ ID NO: 14 |
| Ruminococcaceae Ruminococcaceae_UCG-008 | SEQ ID NO: 15 |
| Lachnospiraceae Tyzzerella | SEQ ID NO: 16 |
| Ruminococcaceae Butyricicoccus | SEQ ID NO: 17 |
| Burkholderiaceae Sutterella | SEQ ID NO: 18 |
| Christensenellaceae Catabacter | SEQ ID NO: 19 |
| Ruminococcaceae Oscillibacter | SEQ ID NO: 20 |
| Veillonellaceae Anaeroglobus | SEQ ID NO: 21 |
| Ruminococcaceae Anaerofilum | SEQ ID NO: 22 |
| Ruminococcaceae Candidatus_Soleaferrea | SEQ ID NO: 23 |
| Lachnospiraceae Oribacterium | SEQ ID NO: 24 |
| Veillonellaceae Allisonella | SEQ ID NO: 25 |
| Listeriaceae Brochothrix | SEQ ID NO: 26 |
| Anaplasmataceae Wolbachia | SEQ ID NO: 27 |
| Enterobacteriaceae Buchnera | SEQ ID NO: 28 |
| Lachnospiraceae Lachnospiraceae_UCG-010 | SEQ ID NO: 29 |
| Burkholderiaceae Alcaligenes | SEQ ID NO: 30 |
| Erysipelotrichaceae Erysipelatoclostridium | SEQ ID NO: 31 |
| Lachnospiraceae Coprococcus_3 | SEQ ID NO: 32 |
| Cardiobacteriaceae Cardiobacterium | SEQ ID NO: 33 |

66. The kit of claim 65, wherein the presence and abundance information of the microorganisms of the one or more genera are detected by detecting the presence and abundance information of a nucleotide sequence having at least 75% of sequence identity to a nucleotide sequence selected from the table or a fragment thereof in the sample.

67. The kit of claim 65, wherein the presence and abundance information of the microorganisms of the one or more genera are detected by detecting the presence and abundance information of a nucleotide sequence having at least 80% of sequence identity to a nucleotide sequence selected from the table or a fragment thereof in the sample.

68. The kit of claim 65, wherein the presence and abundance information of the microorganisms of the one or more genera are detected by detecting the presence and abundance information of a nucleotide sequence having at least 85% of sequence identity to a nucleotide sequence selected from the table or a fragment thereof in the sample.

69. The kit of claim 65, wherein the presence and abundance information of the microorganisms of the one or more genera are detected by detecting the presence and abundance information of a nucleotide sequence having at least 90% of sequence identity to a nucleotide sequence selected from the table or a fragment thereof in the sample.

70. The kit of claim 65, wherein the presence and abundance information of the microorganisms of the one or more genera are detected by detecting the presence and abundance information of a nucleotide sequence having at least 95% of sequence identity to a nucleotide sequence selected from the table or a fragment thereof in the sample.

71. The kit of claim 51, wherein the kit further includes an instruction that describes the method for identifying the subject's responsiveness to immune checkpoint inhibitor therapy through the presence and abundance information of microorganisms of the one or more genera.

72. The kit of claim 71, wherein the method includes identification of the subject's responsiveness to immune checkpoint inhibitor therapy by using a machine learning method.

73. The kit of claim 72, wherein the machine learning method is a random forest model or a logistic regression model.

74. The kit of claim 73, wherein the random forest model or logistic regression model further includes using the presence and abundance information of other types of microorganisms as a feature.

75. The kit of claim 73, wherein the random forest model or logistic regression model further includes using the subject's allergy history as a feature.

76. The kit of claim 51, wherein the subject is identified as responsive or non-responsive to the immune checkpoint inhibitor therapy.

77. The kit of claim 64, wherein the kit further includes a buffer, an enzyme, dNTPs and other components for performing the PCR reaction.
